(19) United States
(12) Patent Application Publication
(10) Pub. No.: US 2008/0176885 A1
(43) Pub. Date: Jul. 24, 2008

(54) Novel synergistic opioid-cannabinoid codrug for pain management

(75) Inventors: Joseph R. Holtman, Lexington, KY (US); Peter A. Crooks, Nicholasville, KY (US); Harpreet K. Dhooper, Lexington, KY (US)

Correspondence Address: Davidson, Davidson & Kappel, LLC, 485 7th Avenue, 14th Floor, New York, NY, 10018, US

(73) Assignee: University of Kentucky, Lexington, KY (US)

(21) Appl. No.: 11/973,818

(22) Filed: Oct. 10, 2007

Related U.S. Application Data
(60) Provisional application No. 60/828,960, filed on Oct. 10, 2006.

(51) Int. Cl. C07D 489/00; A61K 31/439

(52) U.S. Cl. 514/282; 546/44

(57) ABSTRACT

Compounds including an opioid, and a cannabinoid covalently bound by a linker; pharmaceutical formulations including codrugs; methods of manufacture as well as methods of treatment are disclosed.

This application claims priority from U.S. Provisional Patent Application No. 60/828,960, filed Oct. 10, 2006; the disclosure of which is hereby incorporated by reference in its entirety.

### FIELD OF THE INVENTION

The present invention relates to the field of pain management, and more particularly to novel synergistic codrugs.

### BACKGROUND OF INVENTION

There is a continuing need for analgesic medications able to provide high efficacy pain relief while providing more favorable pharmacokinetics and reducing the possibility of undesirable effects. Enhancement of the analgesic effect of opioids with cannabinoids has been described in the art in Enhancement mu opioid antinociception by oral delta 9-tetrahydrocannabinol: dose-response analysis and receptor identification, Cichewicz D, et al., J Pharmacol Exp Ther. 1999 May; 289(2):859-67. Synergy between Δ-9 THC and opioids has also been documented in Antinociceptive synergy between delta(9)-tetrahydrocannabinol and opioids after oral administration, Cichewicz D, J Pharmacol Exp Ther. 2003 March; 304(3):1010-5. However, appropriate dosing of these active agents to the site of action, e.g., the brain or spinal column can be difficult because these drugs exhibit different pharmacokinetics. Therefore, there is a need in the art to devise a way to administer opioids and cannabinoids to provide a more favorable and pharmacokinetic profile.

### OBJECTS OF THE INVENTION

Accordingly, it is an object of the present invention to provide a codrug comprising at least one opioid, at least one cannabinoid, both covalently bound to a linker that is capable of cleavage in the body. Preferably, cleavage occurs at the site of action of these active agents.

It is another object of the present invention to provide a pharmaceutical formulation comprising a codrug comprising a therapeutically effective amount of at least one opioid and a therapeutically effective amount of at least one cannabinoid. In certain other embodiments, the amount of one or both of the active agents is subtherapeutic. In still other embodiments, the active agents provide a synergistic effect.

In accordance with the above objects, it is a further object of the invention to provide a method of preparing a pharmaceutical composition comprising a therapeutically effective amount of a codrug that is capable of being cleaved in the body. In more preferred embodiments, the codrug undergoes cleavage at the site of action of one or both of the active agents.

It is an object of the present invention to provide a method and pharmaceutical formulation (medicament) which allows for reduced plasma concentrations of an opioid analgesic, while still providing effective pain management.

It is a further object of the present invention to provide a method and pharmaceutical formulation (medicament) for effectively treating patients in pain with an opioid analgesic which achieves effective pain management, while at the same time provides the opportunity to reduce side effects, dependence and tolerance which the patients may experience when subjected to prolonged treatment with an opioid.

It is a further object of the present invention to provide a method and pharmaceutical formulation (medicament) for effectively treating patients in pain with an opioid analgesic which achieves prolonged and effective pain management, while at the same time provides the opportunity to reduce side effects, dependence and tolerance which the patients may experience when subjected to prolonged treatment with an opioid.

It is yet a further object to provide a method and pharmaceutical formulation (medicament) for the effective treatment of pain in patients by augmenting the analgesic effect of the opioid.

The present invention is related in part to analgesic pharmaceutical compositions comprising a Cannabinoid together with an opioid analgesic. The opioid analgesic and Cannabinoid can be administered orally, via implant, parenterally, sublingually, rectally, topically, via inhalation, etc. In other embodiments of the invention, the Cannabinoid can be administered separately from the opioid analgesic, as set forth in more detail below.

In certain embodiments, the invention allows for the use of lower doses of the opioid analgesic or the Cannabinoid (referred to as “apparent one-way synergy” herein), or lower doses of both drugs (referred to as “two-way synergy” herein) than would normally be required when either drug is used alone. By using lower amounts of either or both drugs, it is believed that the side effects associated with effective pain management in humans may be significantly reduced.

In certain embodiments, the synergistic combination provides an analgesic effect which is up to about 2-20 times greater than that obtained with the dose of opioid analgesic alone when administered by the oral route. In certain other embodiments, the synergistic combination provides an analgesic effect which is up to about 4-5 times greater than that obtained with the dose of opioid analgesic alone. In such embodiments, the synergistic combinations display what is referred to herein as an “apparent one-way synergy”, meaning that the dose of Cannabinoid synergistically potentiates the effect of the opioid analgesic, but the dose of opioid analgesic does not appear to significantly potentiate the effect of the Cannabinoid. In certain embodiments, the combination is administered in a single dosage form. In other embodiments, the combination is administered separately, preferably concomitantly. In certain embodiments, the synergism exhibited between the Cannabinoid and the opioid analgesic is such that the dosage of opioid analgesic would be sub-therapeutic if administered without the dosage of Cannabinoid. In other embodiments, the present invention relates to a pharmaceutical composition comprising an analgesically effective dose of an opioid analgesic together with a dose of a Cannabinoid effective to augment the analgesic effect of the opioid analgesic.

In certain embodiments, the invention is directed to pharmaceutical formulations comprising a Cannabinoid in an amount sufficient to render a therapeutic effect together with a therapeutically effective or sub-therapeutic amount of an opioid analgesic selected from the group consisting of alfentanil, allylprodine, alphaprodine, anileridine, benzylmorphine, bezitramide, buprenorphine, butorphanol, clonitazene, cyclazocine, desomorphine, dextromoramide, dezocine, diampromide, diamorphone, dihydrocodeine, dihydromorphine, dimenoxadol, dimepheptanol, dimethylthiambutene, dioxaphetylbutyrate, dipipanone, eptazocine, ethoheptazine, ethylmethylthiambutene, ethylmorphine, etonitazene fentanyl, heroin, hydromorphone, hydroxypethidine, isomethadone, ketobemidone, levallorphan, levorphanol, levophenacylmorphan, lofentanil, meperidine, meptazinol, metazocine, methadone, metopon, morphine, myrophine, nalbuphine, narceine, nicomorphine, norlevorphanol, normethadone, nalorphine, normorphine, norpipanone, opium, oxycodone, oxymorphone, papavereturn, pentazocine, phenadoxone, phenomorphan, phenazocine, phenoperidine, piminodine, piritramide, propheptazine, promedol, properidine, propiram, propoxyphene, sufentanil, tilidine, tramadol, salts thereof, complexes thereof; mixtures of any of the foregoing, mixed mu-agonists/antagonists, mu-antagonist combinations, salts or complexes thereof, and the like. In certain embodiments, the opioid analgesic is a mu or kappa opioid agonist. In certain embodiments, the invention is directed to pharmaceutical formulations comprising a Cannabinoid in an amount sufficient to render a therapeutic effect together with a therapeutically effective or sub-therapeutic amount of an opioid analgesic selected from the group consisting of morphine, dihydrocodeine, hydromorphone, oxycodone, oxymorphone, salts thereof, and mixtures of any of the foregoing.

In certain embodiments, the invention is directed to pharmaceutical formulations comprising a Cannabinoid in an amount sufficient to render a therapeutic effect together with a dose of codeine which is analgetic if administered without the Cannabinoid. Such a dose of codeine is from about 5 mg to about 400 mg, preferably from about 15 to about 360 mg, and greater than 20 mg. In other embodiments an equianalgesic dose of another opioid is used as will be appreciated by a clinician selecting an appropriate dose.

The invention further relates to the use of a pharmaceutical combination of a Cannabinoid together with an opioid analgesic to provide effective pain management in humans.

The invention further relates to the use of a Cannabinoid in the manufacture of a pharmaceutical preparation containing a Cannabinoid and an opioid analgesic for the treatment of pain.

The invention further relates to the use of an opioid analgesic in the manufacture of a pharmaceutical preparation containing a Cannabinoid and an opioid analgesic for the treatment of pain.

In a further embodiment of the present invention, the invention comprises an oral solid dosage form comprising an analgesically effective amount of an opioid analgesic together with an amount of a Cannabinoid or pharmaceutically acceptable salt thereof which augments the effect of the opioid analgesic.

Optionally, the oral solid dosage form includes a sustained release carrier which causes the sustained release of the opioid analgesic, or both the opioid analgesic and the Cannabinoid when the dosage form contacts gastrointestinal fluid. The sustained release dosage form may comprise a plurality of substrates which include the drugs. The substrates may comprise matrix spheroids or may comprise inert pharmaceutically acceptable beads which are coated with the drugs. The coated beads are then preferably overcoated with a sustained release coating comprising the sustained release carrier. The matrix spheroid may include the sustained release carrier in the matrix itself; or the matrix may comprise a normal release matrix containing the drugs, the matrix having a coating applied thereon which comprises the sustained release carrier. In yet other embodiments, the oral solid dosage form comprises a tablet core containing the drugs within a normal release matrix, with the tablet core being coated with a sustained release coating comprising the sustained release carrier. In yet further embodiments, the tablet contains the drugs within a sustained release matrix comprising the sustained release carrier. In yet further embodiments, the tablet contains the opioid analgesic within a sustained release matrix and the Cannabinoid coated into the tablet as an immediate release layer.

In certain embodiments of the invention, the pharmaceutical compositions containing the Cannabinoids and opioid drugs set forth herein are administered orally. Such oral dosage forms may contain one or both of the drugs in immediate or sustained release form. For ease of administration, in certain embodiments, the oral dosage form contains both drugs. The oral dosage forms may be in the form of tablets, troches, lozenges, aqueous or oily suspensions, dispersible powders or granules, emulsions, multiparticulate formulations, syrups, elixirs, and the like.

The pharmaceutical compositions containing the cannabinoid and/or the opioid drugs set forth herein may alternatively be in the form of microparticles (e.g., microcapsules, microspheres and the like), which may be injected or implanted into a human patient, or other implantable dosage forms known to those skilled in the art of pharmaceutical formulation. For ease of administration, in certain embodiments, such dosage forms contain both drugs.

Additional pharmaceutical compositions contemplated by the invention further include transdermal dosage forms, suppositories, inhalation powders or sprays, and buccal tablets.

The combination of Cannabinoid and opioid analgesic may further be administered by different routes of administration.

In accordance with the above objects, it is a further object of the invention to provide a compound of the following formula:

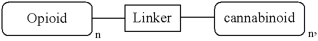

wherein n is an integer from 1 to 5; and the linker is selected from the group consisting of the following formulas:

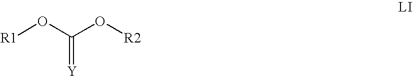

wherein Y is O or S;

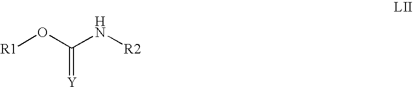

wherein Y is O or S;

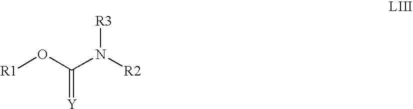

wherein Y is O or S;

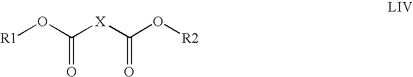

wherein X is a bond or a C₁₋₂₀ alkylene;

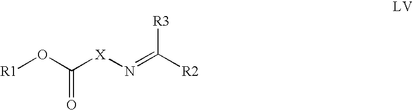

wherein X is a bond or a C₁₋₂₀ alkylene;

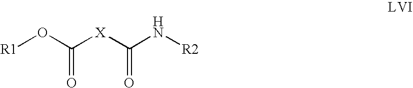

wherein X is a bond or a C₁₋₂₀ alkylene; and

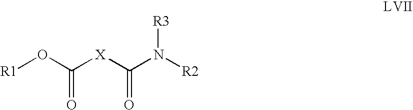

wherein X is a bond or a C₁₋₂₀ alkylene and wherein R1 is a cannabinoid and R2, or R2, R3 are an opioid molecule. In certain embodiments, n is 1 and the linker is C₁₋₄ alkylene. In certain other embodiments, the linker is an alkylene substituted with a heteroatom selected from the group consisting of O and S.

In accordance with the above objects, the invention is also directed to a codrug compound wherein the opioid is Pentazocine, Etorphine, Dihydroetorphine, Phenazocine, Hydrocodone, Methadone, Codeine, Propoxyphene, Meperidine, Morphine, Morphine Sulfate Ester, Tramadol, Hydromorphone, Buprenorphine, Oxymorphone, Levorphanol, L-acetylmethadol, Normethadone, Normorphine, Dihydrocodeine and Ethylmorphine, as well as any pharmaceutically acceptable salts, metabolites, enantiomers, diastereiomers and isomers thereof.

In certain other embodiments, the cannabinoid for combination with the opioid is selected from dronabinol (delta-9-tetrahydrocannabinol) and related cannabinoids such as (−)-delta-9-tetrahydrocannabinol, (+)-delta-9-tetrahydrocannabinoid and delta-8-tetrahydrocannabinol, cannabinol, cannabigerol, cannabicyclol, cannabielsoic acid and their respective pure enantiomers and/or diastereiomers, combinations of the above cannabinoids, plants extracts containing any or all of the above cannabinoids, all naturally occurring cannabinoids, all therapeutically useful and pharmacologically active cannabinoids metabolites, all natural and synthetic nonpsychoactive cannabinoids and their analogs (e.g. dexanabinol), and all psychoactive cannabinoids and their analogs (e.g. nantradol, nabitan) as well as any pharmaceutically acceptable salts, metabolites, enantiomers, diastereiomers and isomers thereof.

In certain preferred embodiments, the codrug compound has the following formula:

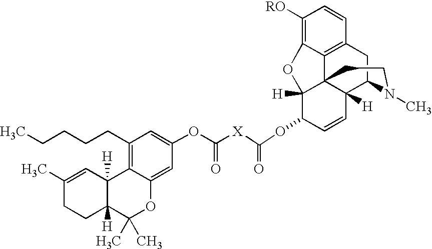

wherein R is H or CH₃; and X is a bond, or alkylene including analogs and stereoisomers thereof.

In still other embodiments of the invention, the codrug compound has the following formula:

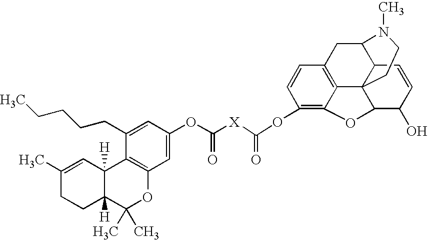

wherein X is a bond or alkylene; including analogs and stereoisomers thereof.

In still other embodiments of the invention, the codrug compound has the following formula:

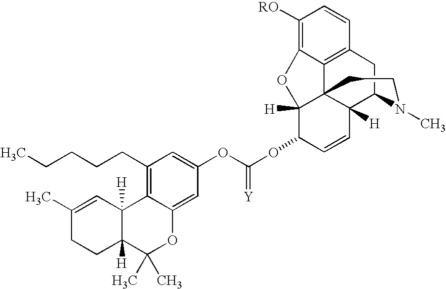

wherein Y is O or S; and R is CH₃; and analogs and stereoisomers thereof.

In still further embodiments of the invention, the codrug compound has the following formula

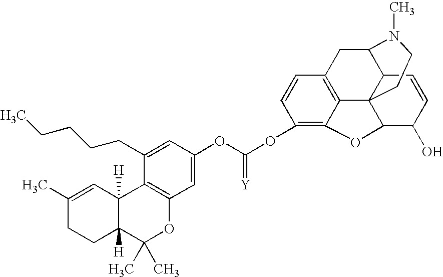

wherein Y is O or S; and analogs and stereoisomers thereof.

In accordance with the above objects, the invention is also directed to pharmaceutical compositions comprising:
an analgesically effective amount of a compound selected from the group consisting of:

### Compound 1:

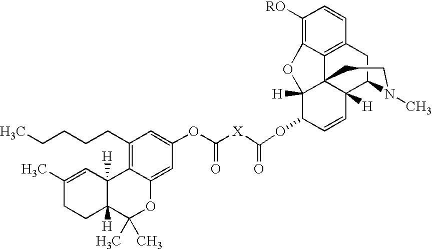

wherein R is H or CH₃; and X is a bond, or alkylene;

### Compound 2:

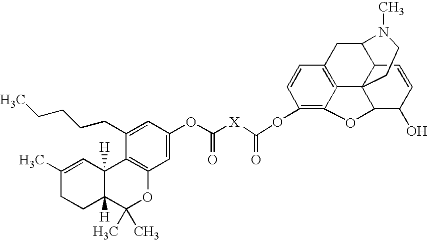

wherein X is a bond or alkylene;

### Compound 3:

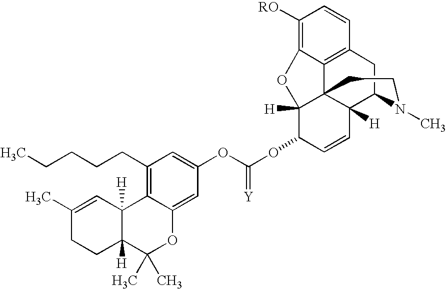

wherein Y is O or S; and R is CH₃; and

### Compound 4:

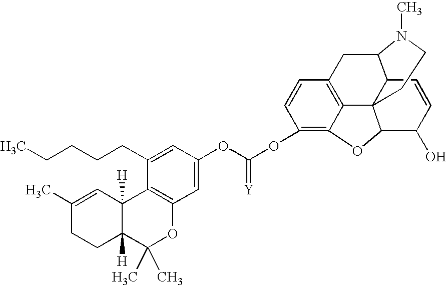

wherein Y is O or S; and at least one pharmaceutically acceptable excipient. In certain embodiments, X is a C₁₋₄ alkylene. In certain other embodiments, R is methyl. In certain other embodiments, R is hydrogen. In still other embodiments, X is a bond.

In certain other embodiments, the invention is directed to pharmaceutical compositions wherein the formulation is suitable for a route of administration selected from the group consisting of: oral, sublingual, oral inhalation, nasal inhalation, sublingual, rectal, vaginal, urethral, intravenous, intraarterial, intradermal, intramuscular, subcutaneous, transdermal, mucosal and buccal.

In accordance with the above objects, the invention is also directed to pharmaceutical compositions wherein the release of the codrug is substantially controlled over an extended period of time selected from the group consisting of: about 4 hours, about 8 hours, about 12 hours, about 18 hours, about 24 hours, about 36 hours, about 48 hours, about 72 hours and about 96 hours. In certain preferred embodiments, the release of the codrug is substantially controlled for about 6-12 hours. In certain other preferred embodiments, the codrug is substantially controlled for about 12-24 hours.

In accordance with the above objects, it is yet a further object of the invention to provide a method of synthesis of a codrug comprising a linker, an opioid and a cannabinoid, wherein the method comprises: covalently bonding a first attachment point of the linker to the opioid; covalently bonding a second attachment point of the linker to a cannabinoid; and recovering the codrug. In certain preferred embodiments, the method comprises reacting para-nitrophenyl chloroformate with an opiate (opioid) (R1) containing a hydroxy group in the presence of triethyl amine and dry chloroform; cooling the solution; recovering the resulting 6-O-para-nitrophenoxycarbonate ester of an opiate drug; reacting the 6-O-para-nitrophenoxycarbonate ester of an opiate drug with a cannabinoid drug (R₂) with a hydroxyl group; recovering the cannabinoid-opioid codrug.

In certain other preferred embodiments, the method comprises: reacting para-nitrophenyl chloroformate with codeine to produce the para-nitrophenoxycarbonate ester of codeine; reacting the para-nitrophenoxycarbonate ester of codeine with Δ-9 THC to produce 6-O, 1-O carbonate linked codrug of codeine and Δ-9 THC. In certain preferred embodiments, reacting para-nitrophenyl chloroformate with codeine to produce the para-nitrophenoxycarbonate ester of codeine occurs in the presence of dry chloroform and triethylamine. In certain other preferred embodiments, the reaction occurs under cooled conditions and in a nitrogen atmosphere.

In still other preferred embodiments, reacting the para-nitrophenoxycarbonate ester of codeine with Δ-9 THC to produce 6-O, 1-O carbonate linked codrug of codeine and Δ-9 THC occurs in the presence of dry THF and triethylamine. In still other preferred embodiments, this reaction occurs under cooled conditions and in a nitrogen atmosphere.

The invention also provides methods of treatment comprising: joining an opioid together with a cannabinoid using a linker to form a cleavable codrug; and administering an analgesically effective amount of the codrug to a human patient. In other embodiments, the codrug substantially remains intact until it reaches the site of action of at least the opioid or the cannabinoid. In further embodiments, the codrug is more lipophilic than the opioid molecule. In still other embodiments, the codrug provides for a more desirable pharmacokinetic profile as compared to the opioid or cannabinoid when administered as distinct molecules.

In accordance with the above objects, the invention is also directed to a method of treatment where the amount of the opioid present in the codrug would be subtherapeutic if administered without the cannabinoid. In other embodiments, the amount of the cannabinoid present in the codrug would be subtherapeutic if administered without the opioid. In still further embodiments, the amount of the cannabinoid and the amount of the opioid present in the codrug would each be subtherapeutic if not administered concomitantly. In yet further embodiments, the synergistic analgesic effect of morphine and dronabinol is about 1.5-3 times greater than the extrapolated additive effect of administering morphine and dronabinol alone. In still further objects of the invention, the synergistic analgesic effect of morphine and dronabinol is about 2.5 times greater than the extrapolated additive effect of administering morphine and dronabinol alone.

### SUMMARY OF THE INVENTION

Embodiments of the present invention include novel synergistic opioid-cannabinoid codrug combinations. Additional embodiments include methods of treating and preventing pain in a subject, comprising administration of a codrug combination of the present invention.

An opioid is, generally, any agent that binds to opioid receptors, found principally in the central nervous system and gastrointestinal tract. There are four broad classes of opioids: endogenous opioid peptides, produced in the body; opium alkaloids, such as morphine (the prototypical opioid) and codeine; semi-synthetic opioids such as heroin and oxycodone; and fully synthetic opioids such as pethidine and methadone that have structures unrelated to the opium alkaloids.

Cannabinoids are, generally, a group of chemicals which activate the body's cannabinoid receptors. Before other types were discovered, the term referred to a unique group of secondary metabolites found in the cannabis plant, which are responsible for the plant's peculiar pharmacological effects. Currently, there are three general types of cannabinoids: herbal cannabinoids occur uniquely in the cannabis plant; endogenous cannabinoids are produced in the bodies of humans and other animals; and synthetic cannabinoids are similar compounds produced in a laboratory.

The present invention relates to pharmaceutical compositions and synthetic methods wherein an opioid analgesic (e.g., Morphine) and a cannabinoid [e.g., Delta-9-tetrahydrocannabinol (THC)] are combined to produce a single chemical co-drug entity and administered in amounts to produce a synergistic (supra-additive) analgesic response to pain (acute, chronic and/or cancer-related). In addition, embodiments of the present invention have a slower rate of opioid tolerance development and dependence with diminished clinical side effects than typically observed with conventional opioid only therapy for pain. Typical side effects known to occur following administration of a cannabinoid are also expected to be diminished preferably while retaining the desirable anti-nausea and anti-anorexic properties.

Codrugs of the present invention comprise two different synergistic drugs (opioid and cannabinoid) within a single chemical entity. The two drugs may be connected directly or by means of a cleavable covalent linker (e.g., ester, carbonate, amide, carbamate, etc.) which is cleaved in vivo to regenerate the active drug entities. There are advantages to delivery of two drugs as a single entity versus a physical mixture. These include, for example, improved drug stability as well as improved targeting of drugs to site of action (central nervous system) and more desirable pharmacokinetic properties, in particular for drugs with different physicochemical properties (e.g., differences in lipid solubility).

Specifically it is believed that when different molecules are linked together and administered as a co-drug, these molecules would undergo the same pharmacokinetics prior to cleavage. Specifically, where different molecules have substantially different partition coefficients, absorption across membranes would be the same. Other advantages of administering different molecules as co-drugs is described in Synthesis and Hydrolytic Behavior of Two Novel Tripartate Codrugs of Naltrexone and 6β-Naltrexone with Hydroxybupropion as Potential Alcohol Abuse and Smoking Cessation Agents, Hamad et al., Bioorganic and Medicinal Chemistry, 2006, volume 14, pages 7051-7061; the disclosure of which is hereby incorporated by reference in its entirety.

### BRIEF DESCRIPTION OF THE FIGURES

FIG. 1 is a graph showing synergistic enhancement of the antinociceptive effect of morphine in combination with delta-9-tetrahydrocannabinol in the thermal tail flick test.

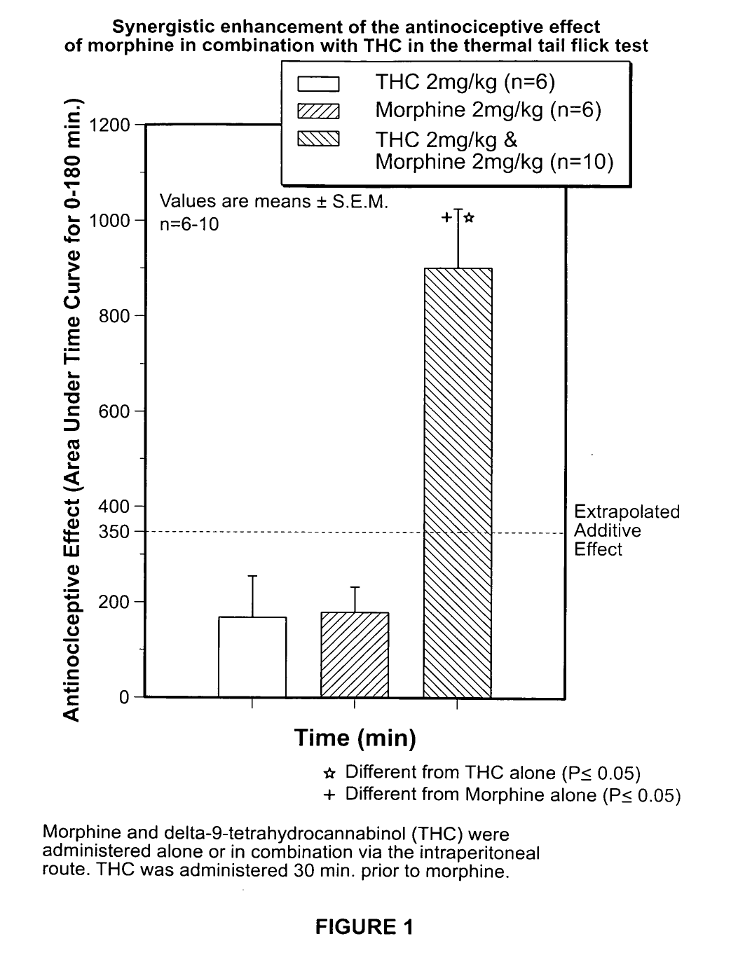

FIG. 2 is a graph showing the time course of the synergistic enhancement of the antinociceptive effect of morphine in combination with delta-9-tetrahydrocannabinol in the thermal tail flick test.

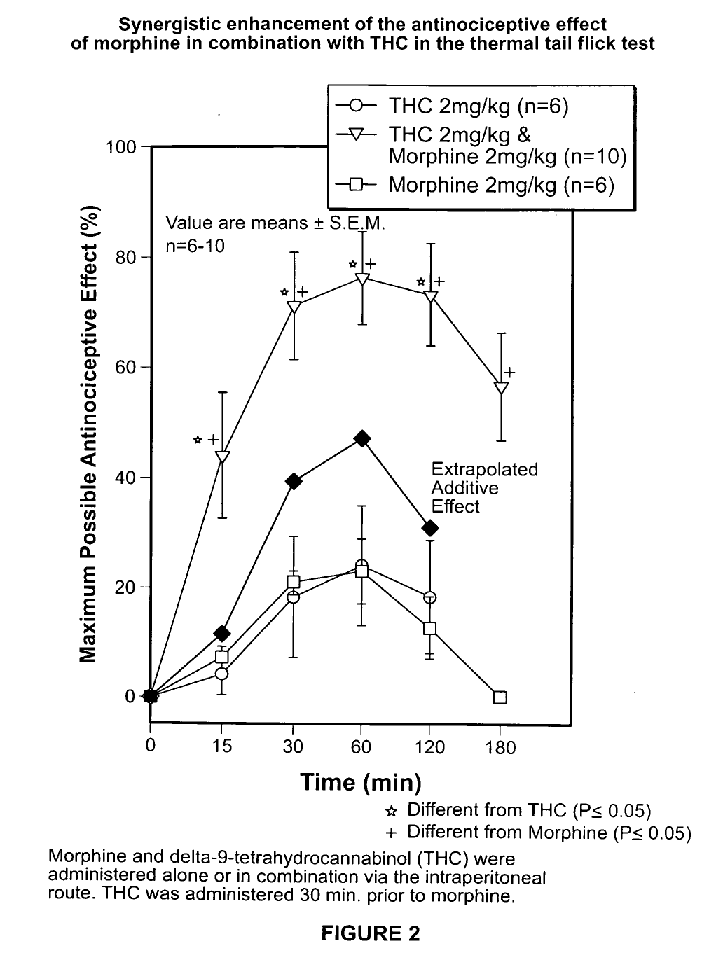

FIG. 3 is depiction of the MALDI analysis of the product of the synthesis described in Example 11.

FIG. 4 is depiction of the MALDI analysis of the product of the synthesis described in Example 12.

### DETAILED DESCRIPTION OF THE INVENTION

As stated above, an aspect of the present invention is novel synergistic opioid-cannabinoid codrug combinations.

One embodiment of the present invention is a composition of the following formula:

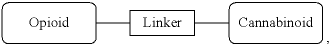

and compositions thereof.

Another embodiment of the present invention is a composition of the following formula:

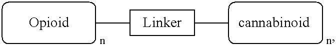

and compositions thereof, wherein n is an integer from 1 to 5.

### Linker

In certain embodiments of the present invention, the linker can be of the following formula wherein R1 is a cannabinoid and R2, or R2, R3 are an opioid molecule:

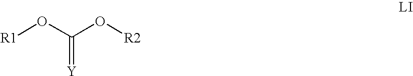

wherein Y is O or S;

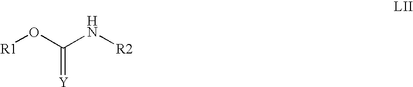

wherein Y is O or S;

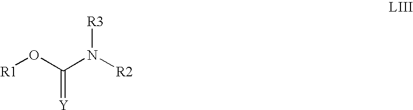

wherein Y is O or S;

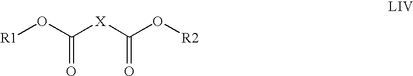

wherein X is a bond or a C₁₋₂₀ alkylene as described herein;

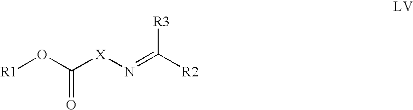

wherein X is a bond or a C₁₋₂₀ alkylene as described herein;

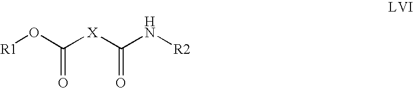

wherein X is a bond or a C₁₋₂₀ alkylene as described herein; or

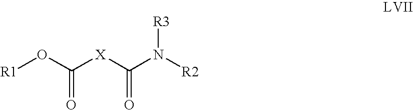

wherein X is a bond or a C₁₋₂₀ alkylene as described herein.

As used herein, (including the claims), the term alkylene or alkylene group is to be understood in the broadest sense to mean hydrocarbon residues which can be linear, i.e., straight-chain, or branched, and can be acyclic or cyclic residues or comprise any combination of acyclic and cyclic subunits. Further, the term alkylene as used herein expressly includes saturated groups as well as unsaturated groups which latter groups contain one or more, for example, one, two, or three, double bonds and/or triple bonds. The term alkylene includes substituted and unsubstituted alkylene groups; one or more carbons may be replaced with heteroatoms O or S; and the alkylene may be pegylated. In accordance with the above substitutions, the alkylene is also understood to include all isomers, diastereiomers, enantiomers; and cis and trans geometrical isomers.

Examples of alkylene residues containing from 1 to 20 carbon atoms are methylene, ethylene, propylene, butylene, pentylene, hexylene, heptylene, octylene, nonylene, decylene, undecylene, dodecylene, tetradecylene, hexadecylene, octadecylene, and eicosylene, the n-isomers of all these residues, isopropylene, isobutylene, 1-methylbutylene, isopentylene, neopentylene, 2,2-dimethylbutylene, 2-methylpentylene, 3-methylpentylene, isohexylene, 2,3,4-trimethylhexylene, isodecylene, sec-butylene, tertbutylene, or tertpentylene. In certain preferred embodiments, the alkylene contains from 1 to 4 carbons.

Unsaturated alkylene residues are, for example, alkenylene residues such as vinylene, 1-propenylene, 2-propenylene (=allyl), 2-butenylene, 3-butenylene, 2-methyl-2-butenylene, 3-methyl-2-butenylene, 5-hexenylene, or 1,3-pentadienylene, or alkynylene residues such as ethynylene, 1-propynylene, 2-propynylene (=propargyl), or 2-butynylene. Alkylene residues can also be unsaturated when they are substituted.

Unless stated otherwise, the term alkylene preferably comprises acyclic saturated hydrocarbon residues containing from 1 to 6 carbon atoms which can be linear or branched. Additionally, included are acyclic unsaturated hydrocarbon residues containing from 2 to 6 carbon atoms which can be linear or branched like (C₂-C₆)-alkenylene and (C₂-C₆)-alkynylene, and cyclic alkylene groups containing from 3 to 8 ring carbon atoms, in particular from 3 to 6 ring carbon atoms. A particular group of saturated acyclic alkylene residues is formed by (C₁-C₄)-alkylene residues like methylene, ethylene, n-propylene, isopropylene, n-butylene, isobutylene, sec-butylene, and tert-butylene.

### Opioids

Examples of opioids for combination with cannabinoids include all therapeutically useful and pharmacologically active opioids and opioid metabolites and their respective pure enantiomers and/or diastereiomers. Representative examples include but are not limited to: Anilopam, Fentanyl, Pentazocine, Dihydroetorphine, Phenazocine, Sufentanil, Codeine, Alfentanil, Meperidine, Morphine, Propoxyphene, Morphine Sulfate Ester, Tramadol, Hydromorphone, Buprenorphine, Oxymorphone, Levorphanol, Methadone, L-acetylmethadol, Oxycodone, Etorphine, Hydrocodone, Normethadone, Remifentanil, Noroxycodone, Dihydrocodeine, Norlevorphanol, Ethylmorphine, Nalbuphine, Hydromorphine, Normorphine, Dihydroetorphine, and Butorphanol.

In certain embodiments, pharmaceutical formulation comprises codeine in an amount of from about 1 to about 400 mg, about 5 to about 360 mg, about 10 mg, about 20 mg, about 30 mg, about 40 mg, about 50 mg, about 60 mg about 60 mg, about 70 mg, about 80 mg, about 90 mg or about 100 mg; or an equianalgesic dose of another opioid.

### Cannabinoids

Representative examples of cannabinoids for combination with opioids include dronabinol (delta-9-tetrahydrocannabinol) and related cannabinoids such as (−)-delta-9-tetrahydrocannabinol, (+)-delta-9-tetrahydrocannabinold and delta-8-tetrahydrocannabinol, cannabinol, cannabigerol, cannabicyclol, cannabielsoic acid and their respective pure enantiomers and/or diastereiomers, combination of the above cannabinoids, plants extracts containing any or all of the above cannabinoids, all naturally occurring cannabinoids, all therapeutically useful and pharmacologically active cannabinoids metabolites, all natural and synthetic nonpsychoactive cannabinoids and their analogs (e.g. dexanabinol), and all psychoactive cannabinoids and their analogs (e.g. nantradol, nabitan). For purposes of the present invention, dronabinol, D-9 tetrahydrocannabinol, Δ-9 tetrahydrocannabinol and Δ-9 THC are synonymous.

In certain embodiments, pharmaceutical formulation comprises Δ-9 THC in an amount of from about 0.1 to about 200 mg, about 0.5 to about 150 mg, about 1 mg, about 2 mg, about 3 mg, about 4 mg, about 5 mg, about 6 mg about 7 mg, about 8 mg, about 9 mg, about 10 mg, about 15 mg, about 20 mg, about 25 mg, about 30 mg, about 35 mg, about 40 mg, about 45 mg, about 50 mg; or an equianalgesic dose of another cannabinoid.

### Synthesis of Codrugs

In certain embodiments, the general multi-step synthetic procedure for preparation of the codrug includes: reacting para-nitrophenyl chloroformate with an opiate drug (R₁) containing a hydroxy group in the presence of triethyl amine and dry chloroform and the solution is cooled to 0° C. The resulting 6-O-para-nitrophenoxycarbonate ester of an opiate drug is then reacted with a cannabinoid drug (R₂) with a hydroxyl group to yield the cannabinoid-opioid codrug.

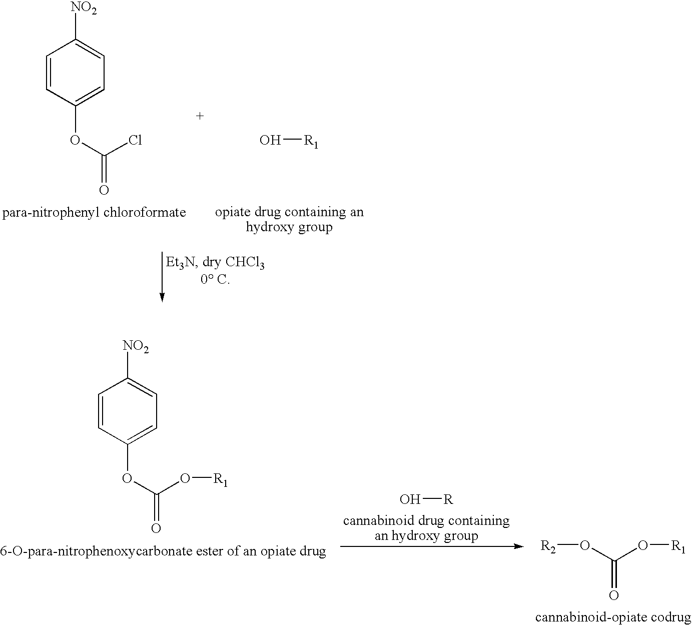

Generally speaking the opioids and cannabinoids of the present invention that are synthesized into co-drugs in accordance with the present invention will contain a free hydroxyl group or another equivalent moiety capable of being acylated. Examples of other moieties include primary or secondary amines, or carbonyl containing moieties. Example of opioids suitable for synthesis of the codrugs in accordance with the present invention include: Pentazocine, Etorphine, Dihydroetorphine, Phenazocine, Hydrocodone, Methadone, Codeine, Propoxyphene, Meperidine, Morphine, Morphine Sulfate Ester, Tramadol, Hydromorphone, Buprenorphine, Oxymorphone, Levorphanol, L-acetylmethadol, Normethadone, Normorphine, Dihydrocodeine and Ethylmorphine, as well as any pharmaceutically acceptable salts, metabolites, enantiomers and diastereiomers thereof.

Examples of cannabinoids suitable for use in the present invention include those recited in U.S. Publication No. 20060160888, the disclosure of which is hereby incorporated by reference in its entirety.

Compositions of the present invention can be synthesized using the methods readily available to the skilled artisan, including those methods known in the art of synthetic organic chemistry, or variations thereon as readily appreciated and readily performable by those skilled in the art. Moreover, the synthesis methods known in the art are not intended to comprise a comprehensive list of all means by which the compositions described and claimed in this patent application may be synthesized.

Some of the compounds of the invention may have stereogenic centers. The compounds may, therefore, exist in at least two and often more stereoisomeric forms. The present invention encompasses all stereoisomers of the compounds whether free from other stereoisomers or admixed with other stereoisomers in any proportion and thus includes, for instance, racemic mixture of enantiomers as well as the diasteriomeric mixture of isomers. Thus, when using the term “compound”, it is understood that all stereoisomers are included.

As used herein, “pharmaceutically effective” and/or “therapeutically effective” amount of a composition of the present invention is an amount that results in a sufficiently high level of pain blockage in an individual or animal.

As used herein, a “mammal” or “individual” refers to humans or animals such as dogs, cats, horses, and the like, and farm animals, such as cows, pigs, guinea pigs and the like.

### Additional Active Agents

According to the formulations and methods of the present invention, the effective compounds described herein may be administered alone or in conjunction with other pharmaceutically active compounds (a.k.a. active agents). It will be understood by those skilled in the art that pharmaceutically active compounds to be used in combination with the compounds described herein will be selected in order to avoid adverse effects on the recipient or undesirable interactions between the compounds. As used herein, the term “active ingredient” or “active agent” is meant to include compounds described herein when used alone or in combination with one or more additional pharmaceutically active compounds. The amount of the compounds described herein required for use in the various treatments of the present invention depend, inter alia, on the route of administration, the age and weight of the animal (e.g. human) to be treated and the severity of the condition being treated.

The compositions of the present invention may be administered in combination with a second therapeutic agent such as, for example, a corticosteroid, etc. The compositions of the present invention and such second therapeutic agent can be administered separately or as a physical combination in a single dosage unit, in any dosage form and by various routes of administration, as described above. The compositions of the present invention may be formulated together with the second therapeutic agent in a single dosage unit (that is, combined together in one liquid, etc.). When the compositions of the present invention and the second therapeutic agent are not formulated together in a single dosage unit, they may be administered essentially at the same time, or in any order; for example, the compositions of the present invention may be administered first, followed by administration of the second agent. When not administered at the same time, preferably the administration of a composition of the present invention and the second therapeutic agent occurs less than about one hour apart, more preferably less than about 5 to 30 minutes apart.

### Salts

The compounds of the present invention may be obtained or used as inorganic or organic salts using methods known to those skilled in the art. It is well known to one skilled in the art that an appropriate salt form is chosen based on physical and chemical stability, flowability, hydroscopicity and solubility. Pharmaceutically acceptable salts of the present invention with an acidic moiety may be optionally formed from organic and inorganic bases. For example with alkali metals or alkaline earth metals such as sodium, potassium, lithium, calcium, or magnesium or organic bases and N-tetraalkylammonium salts such as N-tetrabutylammonium salts. Similarly, when a compound of this invention contains a basic moiety, salts may be optionally formed from organic and inorganic acids.

For example salts may be formed from acetic, propionic, lactic, citric, tartaric, succinic, fumaric, maleic, malonic, mandelic, malic, phthalic, hydrochloric, hydrobromic, phosphoric, nitric, sulfuric, methanesulfonic, naphthalenesulfonic, benzenesulfonic, toluenesulfonic, camphorsulfonic, and similarly known acceptable acids. The compounds can also be used in the form of esters, carbamates and other conventional prodrug forms, which when administered in such form, convert to the active moiety in vivo. When using the term “compound” herein, it is understood that all salts are included.

The term “pharmaceutically acceptable salt” as used herein is intended to include the non-toxic acid addition salts with inorganic or organic acids, e.g. salts with acids such as hydrochloric, phosphoric, sulfuric, maleic, acetic, citric, succinic, benzoic, fumaric, mandelic, p-toluene-sulfonic, methanesulfonic, ascorbic, lactic, gluconic, trifluoroacetic, hydroiodic, hydrobromic, and the like. Examples of pharmaceutically acceptable salts include, but are not limited to, mineral or organic acid salts of basic residues such as amines; alkali or organic salts of acidic residues such as carboxylic acids; and the like.

Pharmaceutically acceptable salts of the compounds of the invention can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, p. 1418, the disclosure of which is hereby incorporated by reference in its entirety.

### Formulations

It is preferred to administer the compounds of the present invention as pharmaceutical formulations. Useful formulations comprise one or more active ingredients and one or more pharmaceutically acceptable carriers. The term “pharmaceutically acceptable” means compatible with the other ingredients of the formulation and not toxic to the recipient. Useful pharmaceutical formulations include those suitable for oral, rectal, nasal, topical, vaginal or parenteral administration, as well as administration by naso-gastric tube. The formulations may conveniently be prepared in unit dosage form and may be prepared by any method known in the art of pharmacy. Such methods include the step of bringing the active ingredient into association with the carrier, which may constitute one or more accessory ingredients. In general, the formulations are prepared by uniformly bringing the active ingredients into association with liquid carriers or finely divided solid carriers or both, and then, if necessary, shaping the product.

### Other Ingredients

In the formulations and methods of the present invention, the inventive compositions can form the active ingredient, and are typically administered in admixture with suitable pharmaceutical diluents, excipients, or carriers (collectively referred to herein as carrier materials) suitably selected. Compositions of the present invention may also be coupled with soluble polymers as targetable drug carriers. Furthermore, the compositions of the present invention may be coupled to a class of biodegradable polymers useful in achieving controlled release of a drug, for example, polylactic acid, polyglycolic acid, copolymers of poly lactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

The present invention accordingly provides a pharmaceutical composition which comprises a compound of this invention in combination or association with a pharmaceutically acceptable carrier. In particular, the present invention provides a pharmaceutical composition which comprises an effective amount of a compound of this invention and a pharmaceutically acceptable carrier.

For pharmaceutical use, the compounds described herein may be taken up in pharmaceutically acceptable carriers, such as, for example, solutions, suspensions, tablets, capsules, ointments, elixirs and injectable compositions. In certain embodiments of the invention, pharmaceutical preparations may contain from 0.1% to 99.9% by weight of active ingredient. Certain examples of preparations in accordance with the present invention which are in single dose form, “unit dosage form”, may contain from 20% to 90% active ingredient, and certain preparations of the present invention which are not in single dose form may contain from 5% to 50% active ingredient. As used herein, the term “active ingredient” refers to compounds described herein, salts thereof and mixtures of compounds described herein with other pharmaceutically active compounds. In certain embodiments of the invention, dosage unit forms such as, for example, tablets or capsules typically contain from about 0.05 to about 1.0 g of active ingredient.

Typically, effective amounts of the compounds of the present invention can range greatly. A skilled artisan or scientist using routine protocols may readily confirm the utility of the compositions described herein.

### Controlled-Release Formulations

Additional pharmaceutical methods may be employed to control the duration of action. Controlled release preparations may be achieved through the use of polymer to complex or absorb the active agents. The controlled delivery may be exercised by selecting appropriate macromolecules (for example polyester, polyamino acids, polyvinyl, pyrrolidone, ethylenevinylacetate, methylcellulose, carboxymethylcellulose, or protamine sulfate) and the concentration of macromolecules as well as the methods of incorporation in order to control release. Alternatively, instead of incorporating these active agents into polymeric particles, it is possible to entrap these active agents in microcapsules prepared, for example, by coacervation techniques or by interfacial polymerization. Other methods and excipients used to created pharmaceutical dosage forms for immediate and modified release of the active agents from the dosage forms in accordance with the present invention are described in U.S. Publication No. 20060160888, the disclosure of which is hereby incorporated by reference in its entirety.

### Coatings

The dosage forms of the present invention may optionally be coated with one or more materials suitable for the regulation of release or for the protection of the formulation. In one embodiment, coatings are provided to permit either pH-dependent or pH-independent release, e.g., when exposed to gastrointestinal fluid. A pH-dependent coating serves to release the opioid in desired areas of the gastro-intestinal (GI) tract, e.g., the stomach or small intestine, such that an absorption profile is provided which is capable of providing at least about twelve hour and preferably up to twenty-four hour analgesia to a patient. When a pH-independent coating is desired, the coating is designed to achieve optimal release regardless of pH-changes in the environmental fluid, e.g., the GI tract. It is also possible to formulate compositions which release a portion of the dose in one desired area of the GI tract, e.g., the stomach, and release the remainder of the dose in another area of the GI tract, e.g., the small intestine.

Formulations according to the invention that utilize pH-dependent coatings to obtain formulations may also impart a repeat-action effect whereby unprotected drug is coated over the enteric coat and is released in the stomach, while the remainder, being protected by the enteric coating, is released further down the gastrointestinal tract. Coatings which are pH-dependent may be used in accordance with the present invention include shellac, cellulose acetate phthalate (CAP), polyvinyl acetate phthalate (PVAP), hydroxypropylmethylcellulose phthalate, and methacrylic acid ester copolymers, zein, and the like.

In certain embodiments, the substrate (e.g., tablet core bead, matrix particle) containing the opioid analgesic (with or without the cannabinoid) is coated with a hydrophobic material selected from (i) an alkylcellulose; (ii) an acrylic polymer; or (iii) mixtures thereof. The coating may be applied in the form of an organic or aqueous solution or dispersion. The coating may be applied to obtain a weight gain from about 2 to about 25% of the substrate in order to obtain a desired sustained release profile. Such formulations are described, e.g., in detail in U.S. Pat. Nos. 5,273,760 and 5,286,493 the disclosures of which are hereby incorporated by reference in their entireties.

Other examples of sustained release formulations and coatings which may be used in accordance with the present invention include U.S. Pat. Nos. 5,324,351; 5,356,467, and 5,472,712, the disclosures of which are hereby incorporated by reference in their entireties.

### Alkylcellulose Polymers

Cellulosic materials and polymers, including alkylcelluloses, provide hydrophobic materials well suited for coating the beads according to the invention. Simply by way of example, one preferred alkylcellulosic polymer is ethylcellulose, although the artisan will appreciate that other cellulose and/or alkylcellulose polymers may be readily employed, singly or in any combination, as all or part of a hydrophobic coating according to the invention.

One commercially-available aqueous dispersion of ethylcellulose is Aquacoat® (FMC Corp., Philadelphia, Pa., U.S.A.). Aquacoat® is prepared by dissolving the ethylcellulose in a water-immiscible organic solvent and then emulsifying the same in water in the presence of a surfactant and a stabilizer. After homogenization to generate submicron droplets, the organic solvent is evaporated under vacuum to form a pseudolatex. The plasticizer is not incorporated in the pseudolatex during the manufacturing phase. Thus, prior to using the same as a coating, it is necessary to intimately mix the Aquacoat® with a suitable plasticizer prior to use.

Another aqueous dispersion of ethylcellulose is commercially available as Surelease® (Colorcon, Inc., West Point, Pa., U.S.A.). This product is prepared by incorporating plasticizer into the dispersion during the manufacturing process. A hot melt of a polymer, plasticizer (dibutyl sebacate), and stabilizer (oleic acid) is prepared as a homogeneous mixture, which is then diluted with an alkaline solution to obtain an aqueous dispersion which can be applied directly onto substrates.

### Acrylic Polymers

In other embodiments of the present invention, the hydrophobic material comprising the controlled release coating is a pharmaceutically acceptable acrylic polymer, including but not limited to acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymer, polyacrylamide, aminoalkyl methacrylate copolymer, poly(methacrylic acid anhydride), and glycidyl methacrylate copolymers.

In certain preferred embodiments, the acrylic polymer is comprised of one or more ammonio methacrylate copolymers. Ammonio methacrylate copolymers are well known in the art, and are described in NF XVII as fully polymerized copolymers of acrylic and methacrylic acid esters with a low content of quaternary ammonium groups.

In order to obtain a desirable dissolution profile, it may be necessary to incorporate two or more ammonio methacrylate copolymers having differing physical properties, such as different molar ratios of the quaternary ammonium groups to the neutral (meth)acrylic esters.

Certain methacrylic acid ester-type polymers are useful for preparing pH-dependent coatings which may be used in accordance with the present invention. For example, there are a family of copolymers synthesized from diethylaminoethyl methacrylate and other neutral methacrylic esters, also known as methacrylic acid copolymer or polymeric methacrylates, commercially available as Eudragit® from Röhm Tech, Inc. There are several different types of Eudragit®. For example, Eudragit® E is an example of a methacrylic acid copolymer which swells and dissolves in acidic media. Eudragit® L is a methacrylic acid copolymer which does not swell at about pH<5.7 and is soluble at about pH>6. Eudragit® S does not swell at about pH<6.5 and is soluble at about pH>7. Eudragit® RL and Eudragit® RS are water swellable, and the amount of water absorbed by these polymers is pH-dependent, however, dosage forms coated with Eudragit® RL and RS are pH-independent.

In certain preferred embodiments, the acrylic coating comprises a mixture of two acrylic resin lacquers commercially available from Rohm Pharma under the Tradenames Eudragit® RL30D and Eudragit® RS30D, respectively. Eudragit® RL30D and Eudragit® RS30D are copolymers of acrylic and methacrylic esters with a low content of quaternary ammonium groups, the molar ratio of ammonium groups to the remaining neutral (meth)acrylic esters being 1:20 in Eudragit® RL30D and 1:40 in Eudragit® RS30D. The mean molecular weight is about 150,000. The code designations RL (high permeability) and RS (low permeability) refer to the permeability properties of these agents. Eudragit® RL/RS mixtures are insoluble in water and in digestive fluids. However, coatings formed from the same are swellable and permeable in aqueous solutions and digestive fluids.

The Eudragit® RL/RS dispersions of the present invention may be mixed together in any desired ratio in order to ultimately obtain a sustained release formulation having a desirable dissolution profile. Desirable sustained release formulations may be obtained, for instance, from a retardant coating derived from 100% Eudragit® RL, 50% Eudragit® RL and 50% Eudragit® RS, and 10% Eudragit® RL:Eudragit® 90% RS. Of course, one skilled in the art will recognize that other acrylic polymers may also be used, such as, for example, Eudragit® L.

### Plasticizers

In embodiments of the present invention where the coating comprises an aqueous dispersion of a hydrophobic material, the inclusion of an effective amount of a plasticizer in the aqueous dispersion of hydrophobic material will further improve the physical properties of the sustained release coating. For example, because ethylcellulose has a relatively high glass transition temperature and does not form flexible films under normal coating conditions, it is preferable to incorporate a plasticizer into an ethylcellulose coating containing sustained release coating before using the same as a coating material. Generally, the amount of plasticizer included in a coating solution is based on the concentration of the film-former, e.g., most often from about 1 to about 50 percent by weight of the film-former. Concentration of the plasticizer, however, can only be properly determined after careful experimentation with the particular coating solution and method of application.

Examples of suitable plasticizers for ethylcellulose include water insoluble plasticizers such as dibutyl sebacate, diethyl phthalate, triethyl citrate, tributyl citrate, and triacetin, although it is possible that other water-insoluble plasticizers (such as acetylated monoglycerides, phthalate esters, castor oil, etc.) may be used. Triethyl citrate is an especially preferred plasticizer for the aqueous dispersions of ethyl cellulose of the present invention.

Examples of suitable plasticizers for the acrylic polymers of the present invention include, but are not limited to citric acid esters such as triethyl citrate NF XVI, tributyl citrate, dibutyl phthalate, and possibly 1,2-propylene glycol. Other plasticizers which have proved to be suitable for enhancing the elasticity of the films formed from acrylic films such as Eudragit® RL/RS lacquer solutions include polyethylene glycols, propylene glycol, diethyl phthalate, castor oil, and triacetin. Triethyl citrate is an especially preferred plasticizer for the aqueous dispersions of ethyl cellulose of the present invention.

It has further been found that the addition of a small amount of talc reduces the tendency of the aqueous dispersion to stick during processing, and acts as a polishing agent.

### Processes for Preparing Coated Beads

When the aqueous dispersion of hydrophobic material is used to coat inert pharmaceutical beads such as nu pareil 18/20 beads, a plurality of the resultant stabilized solid controlled release beads may thereafter be placed in a gelatin capsule in an amount sufficient to provide an effective controlled release dose when ingested and contacted by an environmental fluid, e.g., gastric fluid or dissolution media.

The stabilized controlled release bead formulations of the present invention slowly release the therapeutically active agent, e.g., when ingested and exposed to gastric fluids, and then to intestinal fluids. The controlled release profile of the formulations of the invention can be altered, for example, by varying the amount of overcoating with the aqueous dispersion of hydrophobic material, altering the manner in which the plasticizer is added to the aqueous dispersion of hydrophobic material, by varying the amount of plasticizer relative to hydrophobic material, by the inclusion of additional ingredients or excipients, by altering the method of manufacture, etc. The dissolution profile of the ultimate product may also be modified, for example, by increasing or decreasing the thickness of the retardant coating.

Spheroids or beads coated with a therapeutically active agent are prepared, e.g., by dissolving the therapeutically active agent in water and then spraying the solution onto a substrate, for example, nu pareil 18/20 beads, using a Wuster insert. Optionally, additional ingredients are also added prior to coating the beads in order to assist the binding of the opioid to the beads, and/or to color the solution, etc. For example, a product which includes hydroxypropylmethylcellulose, etc. with or without colorant (e.g., Opadry®, commercially available from Colorcon, Inc.) may be added to the solution and the solution mixed (e.g., for about 1 hour) prior to application of the same onto the beads. The resultant coated substrate, in this example beads, may then be optionally overcoated with a barrier agent, to separate the therapeutically active agent from the hydrophobic controlled release coating. An example of a suitable barrier agent is one which comprises hydroxypropylmethylcellulose. However, any film-former known in the art may be used. It is preferred that the barrier agent does not affect the dissolution rate of the final product.

The beads may then be overcoated with an aqueous dispersion of the hydrophobic material. The aqueous dispersion of hydrophobic material preferably further includes an effective amount of plasticizer, e.g. triethyl citrate. Pre-formulated aqueous dispersions of ethylcellulose, such as Aquacoat® or Surelease®, may be used. If Surelease® is used, it is not necessary to separately add a plasticizer. Alternatively, pre-formulated aqueous dispersions of acrylic polymers such as Eudragit® can be used.

The coating solutions of the present invention preferably contain, in addition to the film-former, plasticizer, and solvent system (i.e., water), a colorant to provide elegance and product distinction. Color may be added to the solution of the therapeutically active agent instead, or in addition to the aqueous dispersion of hydrophobic material. For example, color be added to Aquacoat® via the use of alcohol or propylene glycol based color dispersions, milled aluminum lakes and opacifiers such as titanium dioxide by adding color with shear to water soluble polymer solution and then using low shear to the plasticized Aquacoat®. Alternatively, any suitable method of providing color to the formulations of the present invention may be used. Suitable ingredients for providing color to the formulation when an aqueous dispersion of an acrylic polymer is used include titanium dioxide and color pigments, such as iron oxide pigments. The incorporation of pigments, may, however, increase the retard effect of the coating.

The plasticized aqueous dispersion of hydrophobic material may be applied onto the substrate comprising the therapeutically active agent by spraying using any suitable spray equipment known in the art. In a preferred method, a Wurster fluidized-bed system is used in which an air jet, injected from underneath, fluidizes the core material and effects drying while the acrylic polymer coating is sprayed on. A sufficient amount of the aqueous dispersion of hydrophobic material to obtain a predetermined controlled release of said therapeutically active agent when said coated substrate is exposed to aqueous solutions, e.g. gastric fluid, is preferably applied, taking into account the physical characteristics of the therapeutically active agent, the manner of incorporation of the plasticizer, etc. After coating with the hydrophobic material, a further overcoat of a film-former, such as Opadry®, is optionally applied to the beads. This overcoat is provided, if at all, in order to substantially reduce agglomeration of the beads.

The release of the therapeutically active agent from the controlled release formulation of the present invention can be further influenced, i.e., adjusted to a desired rate, by the addition of one or more release-modifying agents, or by providing one or more passageways through the coating. The ratio of hydrophobic material to water soluble material is determined by, among other factors, the release rate required and the solubility characteristics of the materials selected.

The release-modifying agents which function as pore-formers may be organic or inorganic, and include materials that can be dissolved, extracted or leached from the coating in the environment of use. The pore-formers may comprise one or more hydrophilic materials such as hydroxypropylmethylcellulose.

The sustained release coatings of the present invention can also include erosion-promoting agents such as starch and gums.

The sustained release coatings of the present invention can also include materials useful for making microporous lamina in the environment of use, such as polycarbonates comprised of linear polyesters of carbonic acid in which carbonate groups reoccur in the polymer chain.

The release-modifying agent may also comprise a semi-permeable polymer.

In certain preferred embodiments, the release-modifying agent is selected from hydroxypropylmethylcellulose, lactose, metal stearates, and mixtures of any of the foregoing.

The sustained release coatings of the present invention may also include an exit means comprising at least one passageway, orifice, or the like. The passageway may be formed by such methods as those disclosed in U.S. Pat. Nos. 3,845,770; 3,916,889; 4,063,064; and 4,088,864 (the disclosure of which are hereby incorporated by reference in their entireties). The passageway can have any shape such as round, triangular, square, elliptical, irregular, etc.

### Matrix Bead Formulations

In other embodiments of the present invention, the controlled release formulation is achieved via a matrix having a controlled release coating as set forth above. The present invention may also utilize a controlled release matrix that affords in-vitro dissolution rates of the opioid within the preferred ranges and that releases the opioid in a pH-dependent or pH-independent manner. The materials suitable for inclusion in a controlled release matrix will depend on the method used to form the matrix.

For example, a matrix in addition to the opioid analgesic and (optionally) cannabinoid may include:

Hydrophilic and/or hydrophobic materials, such as gums, cellulose ethers, acrylic resins, protein derived materials; the list is not meant to be exclusive, and any pharmaceutically acceptable hydrophobic material or hydrophilic material which is capable of imparting controlled release of the active agent and which melts (or softens to the extent necessary to be extruded) may be used in accordance with the present invention.

Digestible, long chain (C8-C50, especially C12 C40), substituted or unsubstituted hydrocarbons, such as fatty acids, fatty alcohols, glyceryl esters of fatty acids, mineral and vegetable oils and waxes, and stearyl alcohol; and polyalkylene glycols.

Of these polymers, acrylic polymers, especially Eudragit® RSPO—the cellulose ethers, especially hydroxyalkylcelluloses and carboxyalkylcelluloses, are preferred. The oral dosage form may contain between 1% and 80% (by weight) of at least one hydrophilic or hydrophobic material.

When the hydrophobic material is a hydrocarbon, the hydrocarbon preferably has a melting point of between 25 and 90° C. Of the long chain hydrocarbon materials, fatty (aliphatic) alcohols are preferred. The oral dosage form may contain up to 60% (by weight) of at least one digestible, long chain hydrocarbon.

In certain embodiments, the oral dosage form contains up to 60% (by weight) of at least one polyalkylene glycol.

The hydrophobic material is preferably selected from the group consisting of alkylcelluloses, acrylic and methacrylic acid polymers and copolymers, shellac, zein, hydrogenated castor oil, hydrogenated vegetable oil, or mixtures thereof. In certain embodiments of the present invention, the hydrophobic material is a pharmaceutically acceptable acrylic polymer, including but not limited to acrylic acid and methacrylic acid copolymers, methyl methacrylate, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamine copolymer, poly(methyl methacrylate), poly(methacrylic acid) (anhydride), polymethacrylate, polyacrylamide, poly(methacrylic acid anhydride), and glycidyl methacrylate copolymers. In other embodiments, the hydrophobic material is selected from materials such as hydroxyalkylcelluloses such as hydroxypropylmethylcellulose and mixtures of the foregoing.

Preferred hydrophobic materials are water-insoluble with more or less pronounced hydrophilic and/or hydrophobic trends. Preferably, the hydrophobic materials useful in the invention have a melting point from about 30 to about 200° C., preferably from about 45 to about 90° C. Specifically, the hydrophobic material may comprise natural or synthetic waxes, fatty alcohols (such as lauryl, myristyl, stearyl, cetyl or preferably cetostearyl alcohol), fatty acids, including but not limited to fatty acid esters, fatty acid glycerides (mono-, di-, and tri-glycerides), hydrogenated fats, hydrocarbons, normal waxes, stearic aid, stearyl alcohol and hydrophobic and hydrophilic materials having hydrocarbon backbones. Suitable waxes include, for example, beeswax, glycowax, castor wax and carnauba wax. For purposes of the present invention, a wax-like substance is defined as any material which is normally solid at room temperature and has a melting point of from about 30 to about 100° C.

Suitable hydrophobic materials which may be used in accordance with the present invention include digestible, long chain (C8-C50, especially C12 C40), substituted or unsubstituted hydrocarbons, such as fatty acids, fatty alcohols, glyceryl esters of fatty acids, mineral and vegetable oils and natural and synthetic waxes. Hydrocarbons having a melting point of between 25 and 90° C. are preferred. Of the long chain hydrocarbon materials, fatty (aliphatic) alcohols are preferred in certain embodiments. The oral dosage form may contain up to 60% (by weight) of at least one digestible, long chain hydrocarbon.

Preferably, a combination of two or more hydrophobic materials are included in the matrix formulations. If an additional hydrophobic material is included, it is preferably selected from natural and synthetic waxes, fatty acids, fatty alcohols, and mixtures of the same. Examples include beeswax, carnauba wax, stearic acid and stearyl alcohol. This list is not meant to be exclusive.

One particular suitable matrix comprises at least one water soluble hydroxyalkyl cellulose, at least one C12 C36, preferably C14 C22, aliphatic alcohol and, optionally, at least one polyalkylene glycol. The at least one hydroxyalkyl cellulose is preferably a hydroxy (C1 to C6) alkyl cellulose, such as hydroxypropylcellulose, hydroxypropylmethylcellulose and, especially, hydroxyethylcellulose. The amount of the at least one hydroxyalkyl cellulose in the present oral dosage form will be determined, inter alia, by the precise rate of opioid release required. The at least one aliphatic alcohol may be, for example, lauryl alcohol, myristyl alcohol or stearyl alcohol. In particularly preferred embodiments of the present oral dosage form, however, the at least one aliphatic alcohol is cetyl alcohol or cetostearyl alcohol. The amount of the at least one aliphatic alcohol in the present oral dosage form will be determined, as above, by the precise rate of opioid release required. It will also depend on whether at least one polyalkylene glycol is present in or absent from the oral dosage form. In the absence of at least one polyalkylene glycol, the oral dosage form preferably contains between 20% and 50% (by wt) of the at least one aliphatic alcohol. When at least one polyalkylene glycol is present in the oral dosage form, then the combined weight of the at least one aliphatic alcohol and the at least one polyalkylene glycol preferably constitutes between 20% and 50% (by wt) of the total dosage.

In one embodiment, the ratio of, e.g., the at least one hydroxyalkyl cellulose or acrylic resin to the at least one aliphatic alcohol/polyalkylene glycol determines, to a considerable extent, the release rate of the opioid from the formulation. A ratio of the at least one hydroxyalkyl cellulose to the at least one aliphatic alcohol/polyalkylene glycol of between 1:2 and 1:4 is preferred, with a ratio of between 1:3 and 1:4 being particularly preferred.

The at least one polyalkylene glycol may be, for example, polypropylene glycol or, which is preferred, polyethylene glycol. The number average molecular weight of the at least one polyalkylene glycol is preferred between 1,000 and 15,000 especially between 1,500 and 12,000.

Another suitable controlled release matrix would comprise an alkylcellulose (especially ethyl cellulose), a C12 to C36 aliphatic alcohol and, optionally, a polyalkylene glycol.

In another preferred embodiment, the matrix includes a pharmaceutically acceptable combination of at least two hydrophobic materials.

In addition to the above ingredients, a controlled release matrix may also contain suitable quantities of other materials, e.g. diluents, lubricants, binders, granulating aids, colorants, flavorants and glidants that are conventional in the pharmaceutical art.

### Processes for Preparing Matrix-Based Beads

In order to facilitate the preparation of a solid, controlled release, oral dosage form according to this invention, any method of preparing a matrix formulation known to those skilled in the art may be used. For example incorporation in the matrix may be effected, for example, by (a) forming granules comprising at least one water soluble hydroxyalkyl cellulose and opioid or an opioid salt; (b) mixing the hydroxyalkyl cellulose containing granules with at least one C12-C36 aliphatic alcohol; and (c) optionally, compressing and shaping the granules. Preferably, the granules are formed by wet granulating the hydroxyalkyl cellulose/opioid with water. In a particularly preferred embodiment of this process, the amount of water added during the wet granulation step is preferably between 1.5 and 5 times, especially between 1.75 and 3.5 times, the dry weight of the opioid.

In yet other alternative embodiments, a spheronizing agent, together with the active ingredient can be spheronized to form spheroids. Microcrystalline cellulose is preferred. A suitable microcrystalline cellulose is, for example, the material sold as Avicel PH 101 (Trade Mark, FMC Corporation). In such embodiments, in addition to the active ingredient and spheronizing agent, the spheroids may also contain a binder. Suitable binders, such as low viscosity, water soluble polymers, will be well known to those skilled in the pharmaceutical art. However, water soluble hydroxy lower alkyl celluloses, such as hydroxypropylcellulose, are preferred. Additionally (or alternatively) the spheroids may contain a water insoluble polymer, especially an acrylic polymer, an acrylic copolymer, such as a methacrylic acid ethyl acrylate copolymer, or ethyl cellulose. In such embodiments, the sustained release coating will generally include a hydrophobic material such as (a) a wax, either alone or in admixture with a fatty alcohol; or (b) shellac or zein.

### Melt Extrusion Matrix

Sustained release matrices can also be prepared via melt-granulation or melt-extrusion techniques. Generally, melt-granulation techniques involve melting a normally solid hydrophobic material, e.g. a wax, and incorporating a powdered drug therein. To obtain a sustained release dosage form, it may be necessary to incorporate an additional hydrophobic substance, e.g. ethylcellulose or a water-insoluble acrylic polymer, into the molten wax hydrophobic material. Examples of sustained release formulations prepared via melt-granulation techniques are found in U.S. Pat. No. 4,861,598, the disclosure of which is hereby incorporated by reference in its entirety.

The additional hydrophobic material may comprise one or more water-insoluble wax-like thermoplastic substances possibly mixed with one or more wax-like thermoplastic substances being less hydrophobic than said one or more water-insoluble wax-like substances. In order to achieve constant release, the individual wax-like substances in the formulation should be substantially non-degradable and insoluble in gastrointestinal fluids during the initial release phases. Useful water-insoluble wax-like substances may be those with a water-solubility that is lower than about 1:5,000 (w/w).

In addition to the above ingredients, a sustained release matrix may also contain suitable quantities of other materials, e.g., diluents, lubricants, binders, granulating aids, colorants, flavorants and glidants that are conventional in the pharmaceutical art. The quantities of these additional materials will be sufficient to provide the desired effect to the desired formulation.

In addition to the above ingredients, a sustained release matrix incorporating melt-extruded multiparticulates may also contain suitable quantities of other materials, e.g. diluents, lubricants, binders, granulating aids, colorants, flavorants and glidants that are conventional in the pharmaceutical art in amounts up to about 50% by weight of the particulate if desired.

Specific examples of pharmaceutically acceptable carriers and excipients that may be used to formulate oral dosage forms are described in the Handbook of Pharmaceutical Excipients, American Pharmaceutical Association (1986), incorporated by reference herein.

### Melt Extrusion Multiparticulates

The preparation of a suitable melt-extruded matrix according to the present invention may, for example, include the steps of blending the opioid analgesic, together with at least one hydrophobic material and preferably the additional hydrophobic material to obtain a homogeneous mixture. The homogeneous mixture is then heated to a temperature sufficient to at least soften the mixture sufficiently to extrude the same. The resulting homogeneous mixture is then extruded to form strands. The extrudate is preferably cooled and cut into multiparticulates by any means known in the art. The strands are cooled and cut into multiparticulates. The multiparticulates are then divided into unit doses. The extrudate preferably has a diameter of from about 0.1 to about 5 mm and provides sustained release of the therapeutically active agent for a time period of from about 8 to about 24 hours.

An optional process for preparing the melt extrusions of the present invention includes directly metering into an extruder a hydrophobic material, a therapeutically active agent, and an optional binder; heating the homogenous mixture; extruding the homogenous mixture to thereby form strands; cooling the strands containing the homogeneous mixture; cutting the strands into particles having a size from about 0.1 mm to about 12 mm; and dividing said particles into unit doses. In this aspect of the invention, a relatively continuous manufacturing procedure is realized.

The diameter of the extruder aperture or exit port can also be adjusted to vary the thickness of the extruded strands. Furthermore, the exit part of the extruder need not be round; it can be oblong, rectangular, etc. The exiting strands can be reduced to particles using a hot wire cutter, guillotine, etc.

The melt extruded multiparticulate system can be, for example, in the form of granules, spheroids or pellets depending upon the extruder exit orifice. For purposes of the present invention, the terms “melt-extruded multiparticulate(s)” and “melt-extruded multiparticulate system(s)” and “melt-extruded particles” shall refer to a plurality of units, preferably within a range of similar size and/or shape and containing one or more active agents and one or more excipients, preferably including a hydrophobic material as described herein. In this regard, the melt-extruded multiparticulates will be of a range of from about 0.1 to about 12 mm in length and have a diameter of from about 0.1 to about 5 mm. In addition, it is to be understood that the melt-extruded multiparticulates can be any geometrical shape within this size range. Alternatively, the extrudate may simply be cut into desired lengths and divided into unit doses of the therapeutically active agent without the need of a spheronization step.

In one embodiment, oral dosage forms are prepared to include an effective amount of melt-extruded multiparticulates within a capsule. For example, a plurality of the melt-extruded multiparticulates may be placed in a gelatin capsule in an amount sufficient to provide an effective sustained release dose when ingested and contacted by gastric fluid.

In another preferred embodiment, a suitable amount of the multiparticulate extrudate is compressed into an oral tablet using conventional tableting equipment using standard techniques. Techniques and compositions for making tablets (compressed and molded), capsules (hard and soft gelatin) and pills are also described in Remington's Pharmaceutical Sciences, (Arthur Osol, editor), 1553-1593 (1980), incorporated by reference herein.

In yet another preferred embodiment, the extrudate can be shaped into tablets as set forth in U.S. Pat. No. 4,957,681 (Klimesch, et. al.), the disclosure of which hereby incorporated by reference in its entirety.

Optionally, the sustained release melt-extruded multiparticulate systems or tablets can be coated, or the gelatin capsule can be further coated, with a sustained release coating such as the sustained release coatings described above. Such coatings preferably include a sufficient amount of hydrophobic material to obtain a weight gain level from about 2 to about 30 percent, although the overcoat may be greater depending upon the physical properties of the particular opioid analgesic compound utilized and the desired release rate, among other things.

The melt-extruded unit dosage forms of the present invention may further include combinations of melt-extruded multiparticulates containing one or more of the therapeutically active agents disclosed above before being encapsulated. Furthermore, the unit dosage forms can also include an amount of an immediate release therapeutically active agent for prompt therapeutic effect. The immediate release therapeutically active agent may be incorporated, e.g., as separate pellets within a gelatin capsule, or may be coated on the surface of the multiparticulates after preparation of the dosage forms (e.g., controlled release coating or matrix-based). The unit dosage forms of the present invention may also contain a combination of controlled release beads and matrix multiparticulates to achieve a desired effect.

The sustained release formulations of the present invention preferably slowly release the therapeutically active agent, e.g., when ingested and exposed to gastric fluids, and then to intestinal fluids. The sustained release profile of the melt-extruded formulations of the invention can be altered, for example, by varying the amount of retardant, i.e., hydrophobic material, by varying the amount of plasticizer relative to hydrophobic material, by the inclusion of additional ingredients or excipients, by altering the method of manufacture, etc.

In other embodiments of the invention, the melt extruded material is prepared without the inclusion of the therapeutically active agent, which is added thereafter to the extrudate. Such formulations typically will have the therapeutically active agent blended together with the extruded matrix material, and then the mixture would be tableted in order to provide a slow release formulation. Such formulations may be advantageous, for example, when the therapeutically active agent included in the formulation is sensitive to temperatures needed for softening the hydrophobic material and/or the retardant material.

### Carriers/Excipients

When oral preparations are desired, the component may be combined with typical carriers/excipients, such as lactose, sucrose, starch, talc, magnesium stearate, crystalline cellulose, methyl cellulose, carboxymethyl cellulose, glycerin, sodium alginate or gum arabic among others. The only limitation with respect to the carrier is that it does not deleteriously react with the active compound or is not deleterious to the recipient thereof.

### Auxiliary Agents

The pharmaceutical preparations can be sterilized and if desired mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously react with the active compounds.

### Methods of Treatment

The method of the present invention includes administering the effective compounds described herein to people or animals by any route appropriate as determined by one of ordinary skill in the art. Additionally, physiologically acceptable acid addition salts of compounds described herein are also useful in the methods of treating of the present invention.

Other aspects of the present invention relate to methods of inhibiting pain initiation or signaling in a mammal having a painful response. The methods of the present invention generally comprise administering a pharmaceutically or therapeutically effective amount of a composition as described herein to a patient in need of such treatment whereby pain signaling is inhibited. The patient may be a human or non-human mammal. For example, a patient will need treatment when exhibiting a painful response in the course of a disease (e.g., rheumatoid arthritis) or traumatic condition. Such need is determinable by skilled clinicians and investigators in the medical arts. Additionally, the compounds of the present invention may be used as part of a method of managing pain, or preventing pain prior to, for example, a medical procedure.

### Routes of Administration

Suitable routes of administering the pharmaceutical preparations include, for example, oral, rectal, topical (including transdermal, dermal, buccal and sublingual), vaginal, parenteral (including subcutaneous, intramuscular, intravenous, intradermal, intrathecal and epidural) and by naso-gastric tube.

In aspects of the present invention, the compositions may be administered at the site of perceived pain in a topical, subcutaneous or intramuscular form, using dosage forms well known or readily determinable to those of skill in the pharmaceutical arts. The compositions of the present invention can be administered by any means that produces contact of the active agent with the agent's site of action in the body of a mammal, i.e., the site of pain.

The compositions for the present invention can also be administered in intranasal form via topical use of suitable intranasal vehicles.

It will be understood by those skilled in the art that the preferred route of administration will depend upon the condition being treated and may vary with factors such as the condition of the recipient.

### Prophylactic or Therapeutic Use

In certain embodiments, administration of the compositions of the present invention may be for either a prophylactic or therapeutic use. When provided prophylactically, a compound of the present invention is provided in advance of exposure to conditions indicative of the methods of treatment of the present invention. For example, the compounds of the present invention may be used in advance of a medical procedure believed to produce a pain response.

### Dosing Regimen

The dosage regimen for the compositions of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. An ordinarily skilled physician or veterinarian can readily determine and prescribe the effective amount of the drug required to prevent, counter, or arrest the progress of the painful condition. In certain embodiments, dosage forms (pharmaceutical compositions) suitable for administration may contain from about 1 milligram to about 400 milligrams of active ingredient per dosage unit. In certain other embodiments, dosage forms (pharmaceutical compositions) suitable for administration may contain from about 1 milligram to about 100 milligrams of active ingredient per dosage unit. In certain embodiments, dosage forms (pharmaceutical compositions) suitable for administration may contain from about 10 milligram to about 50 milligrams of active ingredient per dosage unit. In these pharmaceutical compositions the active ingredient will be present in an amount of about 0.1-99.9% by weight based on the total weight of the composition. In certain other embodiments, these pharmaceutical compositions the active ingredient will preferably be present in an amount of about 0.5-95% by weight based on the total weight of the composition. Advantageously, compositions of the present invention may be administered in a single daily dose, or the total daily dosage may be administered in divided doses of two, three, four times or more daily, as needed.

The dosage when administered alone or in combination with a second therapeutic agent may vary depending upon various factors such as the pharmacodynamic characteristics of the particular agent and its mode and route of administration, the age, health and weight of the recipient, the nature and extent of the symptoms, the kind of concurrent treatment, the frequency of treatment, and the effect desired, as described above. The proper dosage of a composition of the present invention when administered in combination with the second therapeutic agent will be readily ascertainable by a medical practitioner skilled in the art, once armed with the present disclosure.

### Maintenance Dose

Upon improvement of a patient's condition, a maintenance dose of a composition of the present invention may be administered, if necessary. Subsequently, the dosage or frequency of administration, or both, may be reduced, as a function of the symptoms, to a level at which the improved condition is retained. When the symptoms have been alleviated to the desired level, treatment should cease. Patients may, however, require intermittent treatment on a long-term basis upon any recurrence of pain.

The following compounds are presented for exemplary purposes only, and should not be construed as being a limited presentation of compounds of the present invention.

### Compound 1:

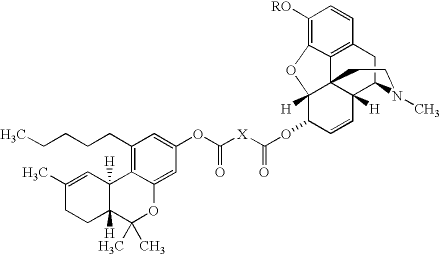

wherein R is H or CH₃; and X is a bond, or alkylene.

### Compound 2:

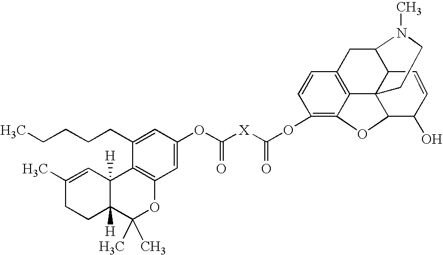

wherein X is a bond or alkylene.

### Compound 3:

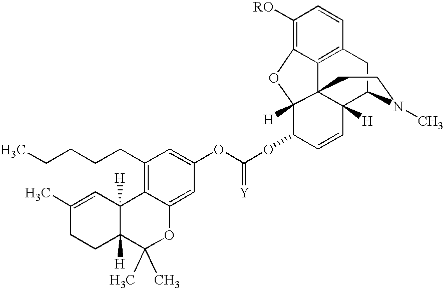

wherein Y is O or S; and R is CH₃.

### Compound 4:

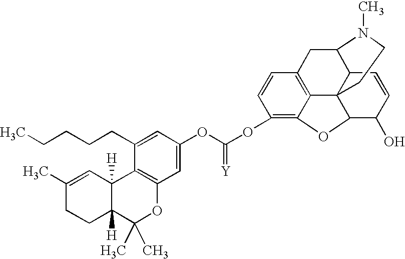

### Y is O or S.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the Specification and Examples be considered as exemplary only, and not intended to limit the scope and spirit of the invention.

Unless otherwise indicated, all numbers expressing quantities of ingredients, properties such as reaction conditions, and so forth used in the Specification and Claims are to be understood as being modified in all instances by the term “about.” Accordingly, unless indicated to the contrary, the numerical parameters set forth in the Specification and Claims are approximations that may vary depending upon the desired properties sought to be determined by the present invention.

Notwithstanding that the numerical ranges and parameters setting forth the broad scope of the invention are approximations, the numerical values set forth in the experimental or example sections are reported as precisely as possible. Any numerical value, however, inherently contain certain errors necessarily resulting from the standard deviation found in their respective testing measurements.

### DETAILED DESCRIPTION OF PREFERRED EMBODIMENTS

### Example 1

### Dose-Response Effect of Morphine, THC and Morphine/THC Combination on Thermally-Induced Nociception Utilizing Tail-Flick Test in Rats

The purpose of the study was to determine the analgesic effect of tetrahydrocannabinol (Δ-9 THC) alone, Morphine Alone and Δ-9 THC in combination with Morphine on thermal-induced pain was determined. The dose-response effect of morphine, Δ-9 THC and morphine-Δ-9 THC combination on thermally-induced nociception utilizing the tail flick test in rats was studied. The dose response curve for the tail flick test and the analgesic effects of various doses of Morphine alone, Δ-9 THC alone as well as various doses of Δ-9 THC combined with various doses of morphine was determined by comparing pre-injection baseline values to post-injection values.

### Tail-Flick Test

The tail-flick test was performed according to the following procedure:

Male Sprague-Dawley rats all with an approximate age of 85 to 90 days were each weighed prior to being subjected to any tests, on the day of the experiment. Each rat was only used for one day of experiments, and given only one dose, or dose combination.
1. Prior to tail-flick test, rats are habituated for three days to handling and the tail flick procedure, without heat exposure.
2. On the day of the experiment, warm up the tail-flick apparatus (IITC Model 33, Life Science, Woodland Hills, Calif.) for at least 30 minutes.
3. Adjust the intensity of the lamp so that baseline tail-flick latency for rats is equal to approximately 2.0 seconds. In this case the intensity will be set to 40% as this was determined to be the ideal intensity from the intensity response curve.
4. The tail-flick apparatus should be programmed to use a cut off point of 10 seconds to prevent tissue damage to the rats in the case that the tail does not flick.
5. Place rat in a mitten and blacken tail with ink approximately 2 inches in length at 1 inch from the tip of the tail.
6. Place the tail flatly in the groove of the tail-flick apparatus.
7. Push start to begin heat exposure. The lamp will turn off automatically when the tail flicks from the heat source.
8. For each rat, a baseline score is determined prior to injection. TFL (tail flick latency) is measured twice in an approximate 15 minute intervals and an average of the two times determines the baseline.
9. Once the baseline value is determined, TFL is measured, following the injection of drugs, at times 15, 30, 60 and 120 minutes.

### Preparation of Solutions

All solutions were made on the day of the experiment. Not all of the solutions described below were used in every experiment. The drug combinations vary for each experiment. Each particular combination for an experiment is reported in the corresponding table below.

Morphine: 3 mg/kg
a. Injection volume of 1 ml/kg: make up solution of 3 mg/ml saline.
b. For 6 rats 9 mg of morphine should be mixed with 3 ml of saline to give the proper amount of drug needed for 3 mg/kg dose at an injection volume of 1 ml/kg.

Morphine: 2 mg/kg

a. Injection volume of 1 ml/kg: make up solution of 2 mg/ml saline.
b. For 6 rats 6 mg of morphine should be mixed with 3 ml of saline to give the proper amount of drug needed for a 2 mg/kg dose at an injection volume of 1 ml/kg.

Δ-9 THC:

Drug is dissolved in a 10 mg/ml ethanol solution (Stock) that must be mixed with cremophore and saline and then dryed before it can be administered to rats. For this experiment the Δ-9 THC was put into a new solution as follows:
i. Take 5 ml of stock solution and add 0.5 ml cremophore and 9 ml saline.
ii. Next the resulting solution should be dryed under nitrogen until there is a total volume of 10 ml. At this point excess EtOH will be dryed off and the solution will contain 0.5 ml EtOH, 0.5 ml cremophore and 9 ml saline (total volume=0.5+9 ml=10 ml; ratio EtOH:cremophore:saline=1:1:18. Solution (A)=50 mg THC in 10 ml=5 mg/ml.
iii. Take 4 ml solution (A) and add 4 ml vehicle (EtOH:cremophore:saline=1:1:18) solution (B)=2.5 mg/ml.
iv. Take 4 ml solution (B) and add 4 ml vehicle (EtOH:cremophore:saline 1:1:18) Solution (C)=1.25 mg/ml.
v. Take 4 ml solution (C) and add 4 ml vehicle (EtOH:cremophore:saline=1:1:18) Solution (D)=0.625 mg/ml.

### Dose Calculations

The dose calculations based on the body weight of the rats for 8 mg/kg, 4 mg/kg, 2 mg/kg, and 1 mg/kg were performed as follows:

Calculations for Dose 8 mg/kg:

Δ-9 THC dose/rat [mg]=8 [mg/kg]×Body Weight [kg]; Volume/rat [ml]=Dose [mg]×1/drug concentration [ml/mg]

Example: Rat Body Weight=0.4 kg; Dose=8 mg/kg×0.4 kg=3.2 mg; Volume=3.2 mg×1/5 ml/mg=0.64 ml

Minimum 4 ml of solution (A) is needed for completion of dose 8 mg/kg experiment (6 rats×0.64 ml=3.84 ml).

Calculations for Dose 4 mg/kg:

THC dose/rat [mg]=4 [mg/kg]×Body Weight [kg]; Volume/rat [ml]=Dose [mg]×1/drug concentration [ml/mg].

Example: Rat Body Weight=0.4 kg; Dose=4 mg/kg×0.4 kg=1.6 mg; Volume=1.6 mg×1/2.5 ml/mg=0.64 ml.

Minimum 4 ml of solution (B) is needed for completion of dose 4 mg/kg experiment (6 rats×0.64 ml=3.84 ml).

Calculations for Dose 2 mg/kg:

THC dose/rat [mg]=2 [mg/kg]×Body Weight [kg]; Volume/rat [ml]=Dose [mg]×1/drug concentration [ml/mg].

Example: Rat Body Weight=0.4 kg; Dose=2 mg/kg×0.4 kg=0.8 mg; Volume=0.8 mg×1/1.25 ml/mg=0.64 ml.

Minimum 4 ml of solution (C) is needed for completion of dose 2 mg/kg experiment (6 rats×0.64 ml=3.84 ml).

Calculations for Dose 1 mg/kg:

THC dose/rat [mg]=1 [mg/kg]×Body Weight [kg]; Volume/rat [ml]=Dose [mg]×1/drug concentration [ml/mg].

Example: Rat Body Weight=0.4 kg; Dose=1 mg/kg×0.4 kg=0.4 mg; Volume=0.4 mg×1/0.625 ml/mg=0.64 ml.

Minimum 4 ml of solution (D) is needed for completion of dose 1 mg/kg experiment (6 rats×0.64 ml=3.84 ml).

Volume [ml]=Dose [mg/kg]*Body Weight [kg]*1/concentration [mg/ml]

Saline solution (control): is designated as Solution E.

Drug Administration i.p. (Intraperitoneal):

Each animal is given an injection of morphine that is 1 ml/kg body weight, and an injection of THC or control that is varies by dose and the injection volume is calculated as set forth above.

Example: body weight=250 g=0.25 kg=0.25 ml

For each drug to be injected: 1 ml/kg×0.250 kg=0.25 ml injected

### Example 2

### Doses A and B

In Example 2, rats 1-3 were given dose A (saline and 8 mg/kg vehicle) and rats 4-6 were given dose B (3 mg/kg morphine and vehicle). The parameters and results of the tail-flick test are set forth in Table 1 below.

TABLE 1

| | | | Volume of | | | | | | |
|---|---|---|---|---|---|---|---|---|---|
| | | | Injections | Tail Flick Latency | | | | | |
| | | | Saline or | (TFL, seconds) | | | | | |
| | | Weight | Morphine/ | | | 15 | 30 | 60 | 120 |
| Rat | Dose | (g) | Vehicle | Base | Base | min | min | min | min |
| 1 | A | 390 | 0.39 ml/0.62 ml | 2.11 | 1.95 | 2.37 | 1.63 | 2.44 | 2.93 |
| 2 | A | 392 | 0.39 ml/0.63 ml | 2.36 | 1.88 | 1.62 | 1.87 | 2.12 | 1.92 |
| 3 | A | 389 | 0.39 ml/0.62 ml | 2.17 | 2.42 | 2.19 | 1.81 | 2.00 | 2.17 |
| 4 | B | 380 | 0.38 ml/0.61 ml | 2.30 | 1.84 | 2.38 | 3.85 | 5.36 | 4.02 |
| 5 | B | 388 | 0.39 ml/0.62 ml | 2.24 | 2.14 | 2.19 | 2.77 | 4.63 | 4.55 |
| 6 | B | 386 | 0.39 ml/0.62 ml | 2.11 | 1.98 | 2.74 | 5.61 | 4.80 | 3.99 |

### Example 3

### Doses C and D

In Example 3, rats 1-3 were given dose C (saline and 8 mg/kg Δ-9 THC) and rats 4-6 were given dose B (3 mg/kg morphine and 8 mg/kg Δ-9 THC). The parameters and results of the tail-flick test are set forth in Table 2 below.

TABLE 2

| | | | Volume of | | | | | | | | |
|---|---|---|---|---|---|---|---|---|---|---|---|
| | | | Injections | Tail Flick Latency | | | | | | | |
| | | | Saline or | (TFL, seconds) | | | | | | | |
| | | Weight | Morphine/ | | | 15 | 30 | 60 | 120 | 180 | 240 |
| Rat | Dose | (g) | Δ-9 THC | Base | Base | min | min | min | min | min | min |
| 1 | C | 393 | .39/0.62 | 2.24 | 2.08 | 3.60 | 10.0 | 10.0 | 10.0 | 10.0 | 8.98 |
| 2 | C | 394 | .39/0.62 | 2.11 | 2.40 | 6.47 | 10.0 | 10.0 | 10.0 | 10.0 | 7.28 |
| 3 | C | 380 | .38/0.61 | 2.33 | 2.12 | 1.57 | 2.81 | 2.87 | 2.19 | 10.0 | 9.21 |
| 4 | D | 360 | .36/0.58 | 2.07 | 1.98 | 10.0 | 10.0 | 10.0 | 10.0 | 10.0 | 10.0 |
| 5 | D | 385 | .39/0.62 | 1.85 | 1.86 | 10.0 | 10.0 | 10.0 | 10.0 | 10.0 | 10.0 |
| 6 | D | 386 | .39/0.62 | 2.03 | 2.68 | 10.0 | 10.0 | 10.0 | 10.0 | 10.0 | 10.0 |

At 15 minutes, Rat 2 did scream but no tail flick. Also, it is suggested that Rat 3 Dose C was possibly given into the bladder or gastrointestinal tract.

### Example 4

### Doses E and F

In Example 4, rats 1-2 were given dose E (saline and 1 mg/kg Δ-9 THC) and rats 3-5 were given dose F (3 mg/kg morphine and 1 mg/kg Δ-9 THC). The parameters and results of the tail-flick test are set forth in Table 3 below.

TABLE 3

| | | | Volume of | | | | | | |
|---|---|---|---|---|---|---|---|---|---|
| | | | Injections | Tail Flick Latency | | | | | |
| | | | Saline or | (TFL, seconds) | | | | | |
| | | Weight | Morphine/ | | | 15 | 30 | 60 | 120 |
| Rat | Dose | (g) | Δ-9 THC | Base | Base | min | min | min | min |
| 1 | E | 388 | 0.39/0.62 | 2.21 | 2.36 | 2.12 | 2.56 | 3.98 | 4.20 |
| 2 | E | 398 | 0.40/0.64 | 1.77 | 2.41 | 2.03 | 2.15 | 5.07 | 3.09 |
| 3 | F | 401 | 0.40/0.64 | 2.36 | 2.47 | 10.0 | 10.0 | 10.0 | 7.95 |
| 4 | F | 386 | 0.39/0.62 | 1.44 | 2.16 | 2.46 | 3.82 | 4.70 | 3.19 |
| 5 | F | 383 | 0.38/0.61 | 2.40 | 2.07 | 3.96 | 4.20 | 5:84 | 6.83 |

### Example 5

### Doses G and H

In Example 5, rats 1-3 were given dose G (saline and 4 mg/kg Δ-9 THC) and rats 4-6 were given dose H (3 mg/kg morphine and 4 mg/kg Δ-9 THC). The parameters and results of the tail-flick test are set forth in Table 4 below.

TABLE 4

| | | | Volume of | | | | | | |
|---|---|---|---|---|---|---|---|---|---|
| | | | Injections | Tail Flick Latency | | | | | |
| | | | Saline or | (TFL, seconds) | | | | | |
| | | Weight | Morphine/ | | | 15 | 30 | 60 | 120 |
| Rat | Dose | (g) | Δ-9 THC | Base | Base | min | min | min | min |
| 1 | G | 392 | .39/0.63 | 2.09 | 1.99 | 5.64 | 5.89 | 8.76 | 7.08 |
| 2 | G | 390 | .39/0.62 | 2.51 | 2.06 | 2.06 | 5.10 | 6.48 | 7.77 |
| 3 | G | 388 | .39/0.62 | 2.07 | 2.07 | 3.99 | 5.07 | 6.24 | 3.53 |
| 4 | H | 356 | .36/0.57 | 2.71 | 2.16 | 5.16 | 10.0 | 10.0 | 10.0 |
| 5 | H | 390 | .39/0.62 | 2.43 | 2.01 | 4.79 | 9.02 | 10.0 | 10.0 |
| 6 | H | 388 | .39/0.62 | 2.27 | 2.10 | 2.54 | 5.61 | 9.17 | 8.27 |

### Example 6

### Doses X and Y

In Example 6, rats 1-3 were given dose X (saline and 2 mg/kg Δ-9 THC) and rats 4-6 were given dose Y (2 mg/kg morphine and 2 mg/kg Δ-9 THC). The parameters and results of the tail-flick test are set forth in Table 5 below.

TABLE 5

| | | | Volume of | | | | | | |
|---|---|---|---|---|---|---|---|---|---|
| | | | Injections | Tail Flick Latency | | | | | |
| | | | Saline or | (TFL, seconds) | | | | | |
| | | Weight | Morphine/ | | | 15 | 30 | 60 | 120 |
| Rat | Dose | (g) | Δ-9 THC | Base | Base | min | min | min | min |
| 1 | X | 342 | 0.34/0.55 | 1.87 | 1.89 | 1.77 | 5.48 | 6.05 | 5.31 |
| 2 | X | 348 | 0.35/0.56 | 2.23 | 2.06 | 2.10 | 2.86 | 2.58 | 2.04 |
| 3 | X | 347 | 0.35/0.56 | 2.63 | 2.27 | 2.33 | 2.45 | 2.46 | 2.14 |
| 4 | Y | 358 | 0.36/0.57 | 2.67 | 2.46 | 3.83 | 4.53 | 5.02 | 6.04 |
| 5 | Y | 345 | 0.36/0.55 | 2.11 | 2.01 | 2.57 | 4.92 | 4.42 | 6.42 |
| 6 | Y | 359 | 0.36/0.57 | 2.04 | 2.06 | 1.97 | 2.60 | 4.55 | 5.22 |

### Example 7

### Dose Z

In Example 7, rats 1-3 were given dose Z (vehicle and 2 mg/kg morphine). The parameters and results of the tail-flick test are set forth in Table 6 below.

TABLE 6

| | | | Volume of | Tail Flick Latency | | | | | |
|---|---|---|---|---|---|---|---|---|---|
| | | | Injections | (TFL, seconds) | | | | | |
| | | Weight | Morphine/ | | | 15 | 30 | 60 | 120 |
| Rat | Dose | (g) | Vehicle | Base | Base | min | min | min | min |
| 1 | Z | 340 | 0.34/0.54 | 2.12 | 2.45 | 3.08 | 2.96 | 2.22 | 1.84 |
| 2 | Z | 354 | 0.35/0.57 | 2.02 | 2.14 | 3.12 | 2.83 | 3.69 | 4.01 |
| 3 | Z | 337 | 0.34/0.54 | 2.12 | 2.23 | 2.28 | 4.73 | 3.85 | 2.98 |

### Example 8

### Doses X and Y

In Example 8, rats 1-3 were given dose X (saline and 2 mg/kg Δ-9 THC) and rats 4-10 were given dose Y (2 mg/kg morphine and 2 mg/kg Δ-9 THC). The parameters and results of the tail-flick test are set forth in Table 7 below.

TABLE 7

| | | | Volume of | | | | | | |
|---|---|---|---|---|---|---|---|---|---|
| | | | Injections | Tail Flick Latency | | | | | |
| | | | Saline or | (TFL, seconds) | | | | | |
| | | Weight | Morphine/ | | | 15 | 30 | 60 | 120 |
| Rat | Dose | (g) | Δ-9 THC | Base | Base | min | min | min | min |
| 1 | X | 362 | 0.36/0.58 | 2.01 | 2.13 | 1.88 | 1.76 | 2.51 | 1.84 |
| 2 | X | 362 | 0.36/0.58 | 1.58 | 2.17 | 3.65 | 6.75 | 6.89 | 6.24 |
| 3 | X | 397 | 0.40/0.64 | 1.64 | 1.90 | 2.57 | 1.83 | 3.38 | 3.61 |
| 4 | Y | 386 | 0.39/0.62 | 1.83 | 2.52 | 1.72 | 2.02 | 2.97 | 3.44 |
| 5 | Y | 354 | 0.35/0.57 | 1.56 | 1.93 | 4.21 | 9.71 | 10.0 | 10.0 |
| 6 | Y | 370 | 0.37/0.59 | 1.67 | 1.89 | 7.66 | 10.0 | 10.0 | 10.0 |
| 7 | Y | 348 | 0.35/0.56 | 2.22 | 2.30 | 2.01 | 2.69 | 4.22 | 3.19 |
| 8 | Y | 373 | 0.37/0.60 | 1.74 | 2.19 | 2.09 | 2.84 | 2.63 | 4.30 |
| 9 | Y | 371 | 0.37/0.59 | 1.72 | 2.03 | 1.51 | 6.42 | 4.58 | 4.11 |
| 10 | Y | 373 | 0.37/0.60 | 2.16 | 2.01 | 3.08 | 4.85 | 3.51 | 6.58 |

### Example 9

### Dose Z

In Example 9, rats 1-3 were given dose Z (2 mg/kg Morphine and vehicle). The parameters and results of the tail-flick test are set forth in Table 8 below.

TABLE 8

| | | | Volume of | Tail Flick Latency | | | | | |
|---|---|---|---|---|---|---|---|---|---|
| | | | Injections | (TFL, seconds) | | | | | |
| | | Weight | Morphine/ | | | 15 | 30 | 60 | 120 |
| Rat | Dose | (g) | Vehicle | Base | Base | min | min | min | min |
| 1 | Z | 352 | 0.35/0.56 | 1.92 | 2.40 | 2.62 | 4.22 | 2.91 | 2.68 |
| 2 | Z | 364 | 0.36/0.58 | 2.67 | 1.95 | 4.51 | 4.99 | 5.25 | 4.83 |
| 3 | Z | 371 | 0.37/0.59 | 1.80 | 1.95 | 2.01 | 3.00 | 3.97 | 2.67 |

### Example 10

### Doses Y and Z

In Example 10, the purpose of the experiment was to determine the analgesic effect of a 2 mg/kg dose of Δ-9 THC given 30 minutes prior to a 2 mg/kg injection of morphine and also the effects of the control given 30 minutes prior to morphine 2 mg/kg. The determination is made by comparing pre-injection baseline values to post-injection values. Rats 1-10 were given dose Y (2 mg/kg morphine 30 minutes after 2 mg/kg Δ-9 THC) and rats 11-16 were given dose Z (2 mg/kg morphine 30 minutes after vehicle (i.e., control). The parameters and results of the tail-flick test are set forth in Table 9 below.

TABLE 9

| | | | Volume of | | | | | | | |
|---|---|---|---|---|---|---|---|---|---|---|
| | | | Injections | Tail Flick Latency | | | | | | |
| | | | Morphine/ | (TFL, seconds) | | | | | | |
| | | Weight | Δ-9 THC | | | 15 | 30 | 60 | 120 | 180 |
| Rat | Dose | (g) | or vehicle | Base | Base | min | min | min | min | min |
| 1 | Y | 365 | 0.37/0.58 | 2.00 | 2.01 | 2.86 | 3.51 | 6.36 | 5.52 | 3.08 |
| 2 | Y | 369 | 0.37/0.59 | 2.20 | 3.06 | 5.26 | 8.68 | 7.80 | 7.26 | 6.90 |
| 3 | Y | 374 | 0.37/0.60 | 2.60 | 2.98 | 6.60 | 6.91 | 6.26 | 6.14 | 5.05 |
| 4 | Y | 359 | 0.36/0.57 | 2.49 | 2.41 | 2.32 | 5.47 | 6.32 | 6.02 | 5.75 |
| 5 | Y | 360 | 0.36/0.58 | 2.02 | 2.17 | 3.15 | 9.71 | 10.0 | 9.80 | 10.0 |
| 6 | Y | 356 | 0.36/0.57 | 2.44 | 2.04 | 9.06 | 10.0 | 10.0 | 10.0 | 7.91 |
| 7 | Y | 366 | 0.37/0.59 | 2.24 | 2.49 | 3.40 | 5.03 | 5.23 | 4.75 | 3.54 |
| 8 | Y | 368 | 0.37/0.59 | 2.41 | 2.31 | 10.0 | 9.06 | 10.0 | 9.61 | 10.0 |
| 9 | Y | 371 | 0.37/0.59 | 2.78 | 2.32 | 8.47 | 9.62 | 10.0 | 10.0 | 6.89 |
| 10 | Y | 364 | 0.36/0.58 | 2.50 | 2.10 | 6.09 | 10.0 | 10.0 | 10.0 | 7.86 |
| 11 | Z | 353 | 0.35/0.56 | 2.37 | 2.33 | 2.41 | 2.90 | 3.26 | 2.68 | 2.15 |
| 12 | Z | 359 | 0.36/0.57 | 2.48 | 2.16 | 2.99 | 3.58 | 3.14 | 2.94 | 2.64 |
| 13 | Z | 370 | 0.37/0.59 | 2.74 | 2.75 | 3.89 | 4.86 | 3.96 | 3.18 | 2.59 |
| 14 | Z | 358 | 0.36/0.57 | 2.05 | 2.55 | 2.36 | 4.07 | 3.86 | 2.68 | 2.18 |
| 15 | Z | 356 | 0.36/0.57 | 2.74 | 2.41 | 2.97 | 4.39 | 3.99 | 3.06 | 2.56 |
| 16 | Z | 347 | 0.35/0.56 | 2.57 | 2.46 | 3.02 | 3.90 | 4.01 | 3.23 | 2.67 |

### Example 11

### Synthesis of the Para-Nitrophenoxycarbonate Ester of Codeine

In Example 11, the para-nitrophenoxycarbonate ester of codeine is synthesized according to the following schematic.

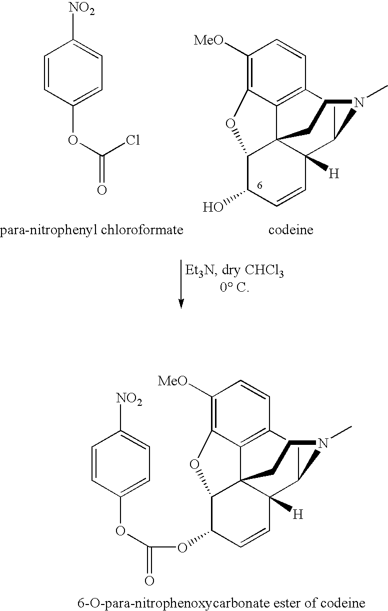

The components and amounts used in this synthesis are set forth in Table 10 below.

TABLE 10

| | Molecular | | | | | |
|---|---|---|---|---|---|---|
| | mass | Wt. | Density | Volume | | |
| Chemicals | (g/mol) | (in g.) | g/mL | (mL) | mmol | Equiv. |
| PNPCF | 201.5 | .037 | | | .18 | 1.1 |
| Codeine | 299 | .05 | | | .16 | 1.0 |
| TEA | 101 | .018 | .726 | .025 | .18 | 1.1 |
| CHCl₃ | | | | app. 5 mL | | |

PNPCF = (para-nitrophenylchloroformate); TEA = (triethylamine)

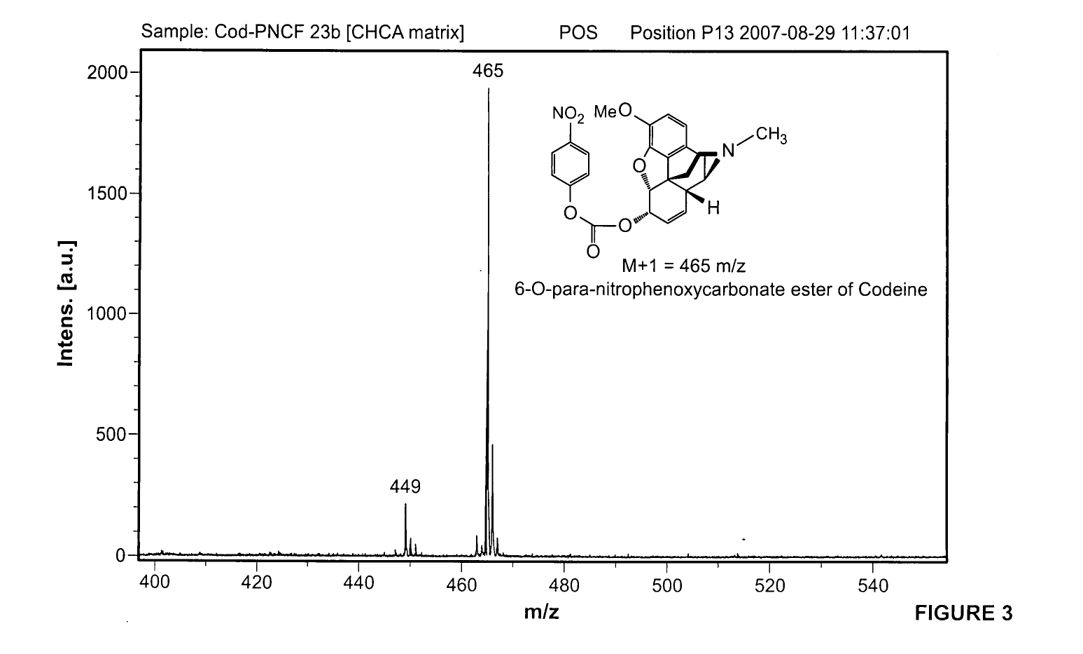

The para-nitrophenoxycarbonate ester of codeine of Example 11 was prepared according to the following procedure. All glassware was oven dried and cooled under a nitrogen atmosphere. 50 mg (0.16 mmol) of codeine was placed in a round-bottom flask under a nitrogen atmosphere and was dissolved in 2 mL of dry chloroform. The solution was cooled down to 0° C. 0.025 mL (0.18 mmol) of triethyl amine was added to the solution drop-wise and the mixture was allowed to stir for 5 minutes. 37 mg (0.18 mmol) of para-nitrophenyl chloroformate was dissolved in 3 mL of dry chloroform and this solution was added to the reaction mixture drop-wise; the reaction mixture was then allowed to warm to room temperature. The progress of the reaction was monitored by TLC. After the reaction was complete, the reaction mixture was concentrated under vacuum to afford an oily residue. A yellowish-white solid was obtained when the oil was treated with hexane. This solid was washed twice with a small volume of hexane and then dissolved in chloroform. The chloroform layer was washed with cold water several times, to remove residual traces of para-nitrophenol. The chloroform layer was then dried over anhydrous sodium sulfate, filtered and then concentrated under vacuum, and the residue washed with hexane to afford the para-nitrophenoxycarbonate ester of codeine as a pale-yellow solid in 35% yield. The MALDI analysis of this synthesis depicted in FIG. 3.

### Example 12

### Synthesis of the 6-O, 1-O Carbonate Linked Codrug of Codeine and Δ-9 THC

In Example 12, the 6-O, 1-O carbonate linked codrug of codeine and Delta-9 THC is synthesized according to the following schematic.

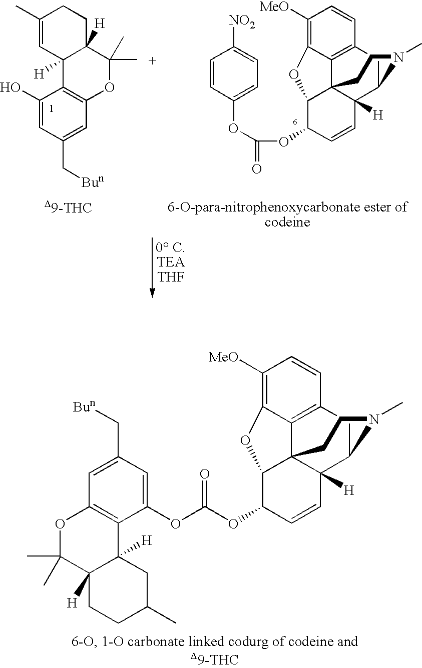

The components and amounts used in this synthesis are set forth in Table 11 below.

TABLE 11

| | Molecular | | | | | |
|---|---|---|---|---|---|---|
| | mass | Wt. | Density | Vol. | | |
| Chemicals | (g/mol) | (in g.) | g/mL | (mL) | mmol | Equiv. |
| Carbonate of | 464 | .056 | | | .12 | 1.0 |
| codeine and PNPCF | | | | | | |
| Δ-9 THC | 314 | .042 | | | .13 | 1.1 |
| TEA | 101 | .013 | .726 | .018 | .13 | 1.1 |
| THF | | | | app. 5 | | |
| (tetrahydrofuran) | | | | mL | | |

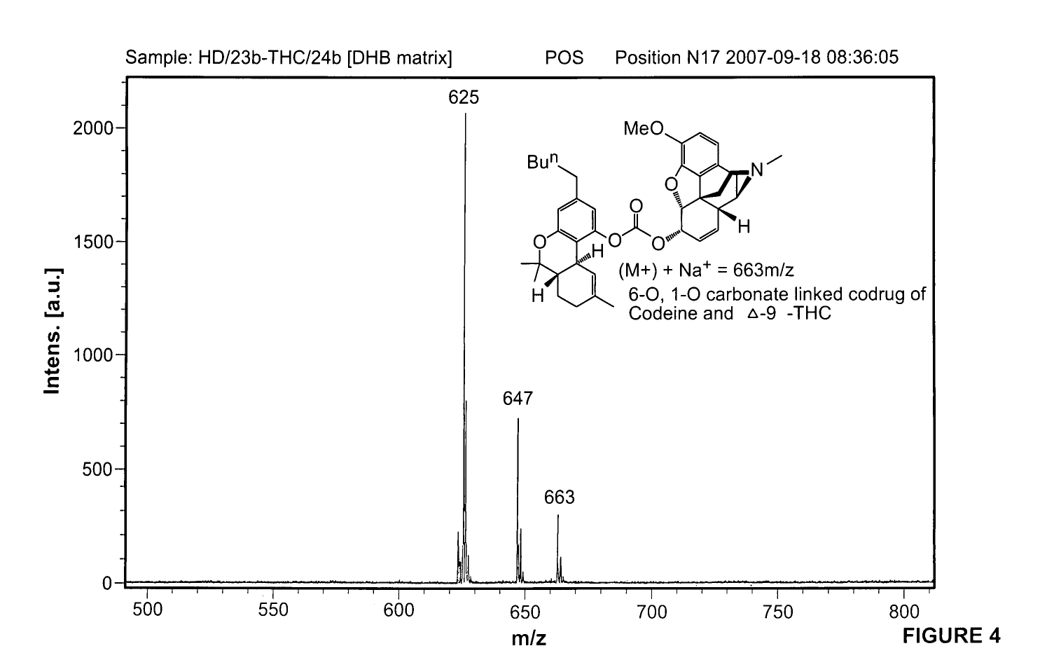

The 6-O, 1-O carbonate linked codrug of codeine and Δ-9 THC of Example 12 was prepared according to the following procedure. All glassware was oven dried and then cooled under a nitrogen atmosphere. 42 mg (0.13 mmol) of Δ-9 THC was placed in a round-bottom flask under a nitrogen atmosphere and dissolved in 2 mL of dry THF (tetrahydrofuran). The solution was cooled to 0° C. 0.018 mL (0.13 mmol) of triethylamine was added to the solution drop-wise and the mixture was allowed to stir for 5 minutes. 56 mg (0.12 mmol) of the para-nitrophenoxycarbonate ester of codeine obtained from the above reaction was dissolved in 3 mL of dry THF and the resulting solution was added to the reaction mixture drop-wise; the reaction mixture was allowed to warm to room temperature. The progress of the reaction was monitored by TLC. After the reaction was complete, the reaction mixture was concentrated under vacuum to afford the 6-O, 1-O carbonate linked codrug of codeine and Δ-9 THC as an amorphous solid in 11% yield. The MALDI analysis of this synthesis is depicted in FIG. 4.

1. A compound of the following formula: 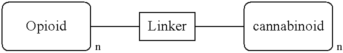
wherein n is an integer from 1 to 5; and the linker is selected from the group consisting of the following formulas: 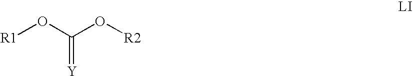
wherein Y is O or S; 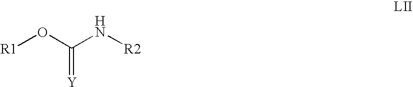
wherein Y is O or S; 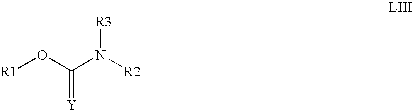
wherein Y is O or S; 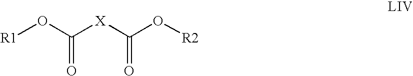
wherein X is a bond or a C₁₋₂₀ alkylene; 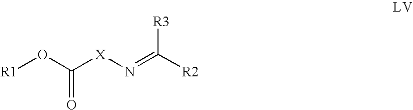
wherein X is a bond or a C₁₋₂₀ alkylene; 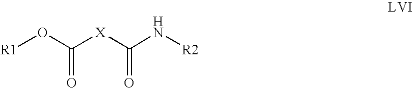
wherein X is a bond or a C₁₋₂₀ alkylene; and 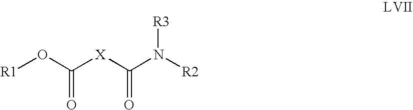
wherein X is a bond or a C₁₋₂₀ alkylene and wherein R1 is a cannabinoid and R2, or R2, R3 are an opioid molecule.

2. The compound of claim 1, wherein n is 1 and the linker is C₁₋₄ alkylene.

3. The compound of claim 1 wherein n is 1; and the linker is an alkylene substituted with a heteroatom selected from the group consisting of O and S.

4. The compound of claim 2, wherein the opioid is Pentazocine, Etorphine, Dihydroetorphine, Phenazocine, Hydrocodone, Methadone, Codeine, Propoxyphene, Meperidine, Morphine, Morphine Sulfate Ester, Tramadol, Hydromorphone, Buprenorphine, Oxymorphone, Levorphanol, L-acetylmethadol, Normethadone, Normorphine, Dihydrocodeine and Ethylmorphine, as well as any pharmaceutically acceptable salts, metabolites, enantiomers, diastereiomers and isomers thereof.

5. The compound of claim 4, wherein the cannabinoid for combination with the opioid is selected from dronabinol (delta-9-tetrahydrocannabinol) and related cannabinoids such as (−)-delta-9-tetrahydrocannabinol, (+)-delta-9-tetrahydrocannabinoid and delta-8-tetrahydrocannabinol, cannabinol, cannabigerol, cannabicyclol, cannabielsoic acid and their respective pure enantiomers and/or diastereiomers, combinations of the above cannabinoids, plants extracts containing any or all of the above cannabinoids, all naturally occurring cannabinoids, all therapeutically useful and pharmacologically active cannabinoids metabolites, all natural and synthetic nonpsychoactive cannabinoids and their analogs (e.g. dexanabinol), and all psychoactive cannabinoids and their analogs (e.g. nantradol, nabitan) as well as any pharmaceutically acceptable salts, metabolites, enantiomers, diastereiomers and isomers thereof.

6. The compound of claim 1 having the following formula: 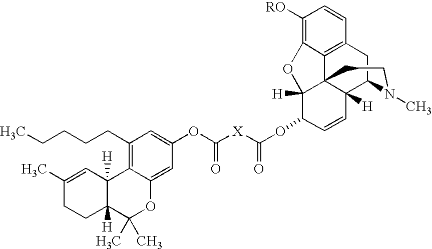
wherein R is H or CH₃; and X is a bond, or alkylene; and analogs and stereoisomers thereof.

7. The compound of claim 1 having the following formula: 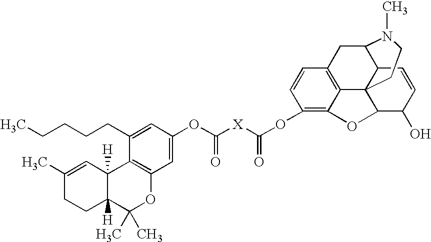
wherein X is a bond or alkylene; and analogs and stereoisomers thereof.

8. The compound of claim 1 having the following formula: 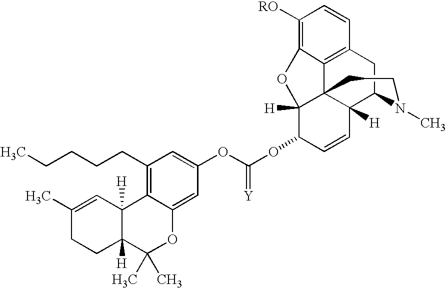
wherein Y is O or S; and R is CH₃; and analogs and stereoisomers thereof.

9. The compound of claim 1 having the following formula 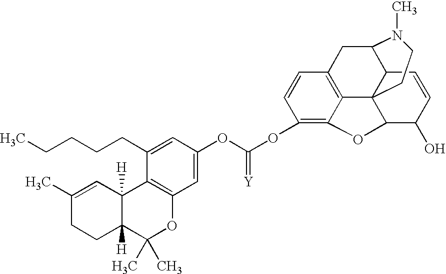
wherein Y is O or S; and analogs and stereoisomers thereof.

10. A pharmaceutical composition comprising:
an analgesically effective amount of a compound selected from the group consisting of:
Compound 1: 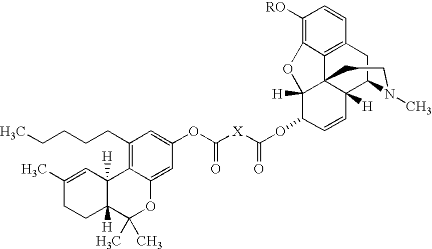
wherein R is H or CH₃; and X is a bond, or alkylene;
Compound 2: 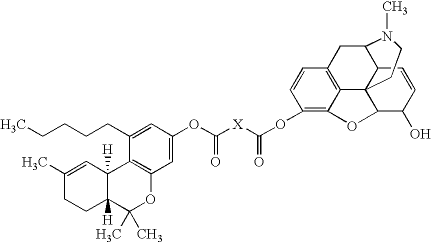
wherein X is a bond or alkylene;
Compound 3: 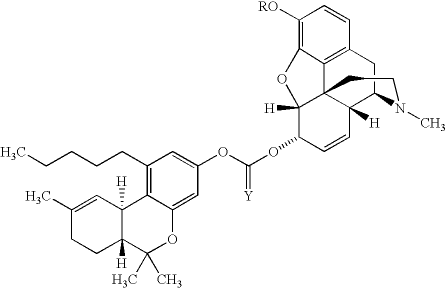
wherein Y is O or S; and R is CH₃; and
Compound 4: 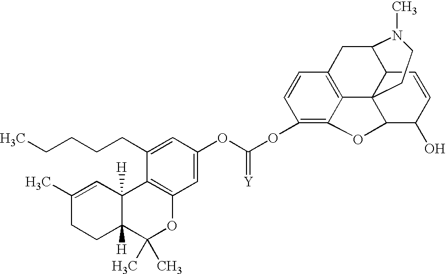
Y is O or S; and at least one pharmaceutically acceptable excipient.

11. The pharmaceutical composition of claim 10 wherein the compound is Compound 1 or Compound 2; and X is a C₁₋₄ alkylene.

12. The pharmaceutical composition of claim 10 wherein the compound is Compound 1 and R is methyl.

13. The pharmaceutical composition of claim 10 wherein the compound is Compound 1 and R is hydrogen.

14. The pharmaceutical composition of claim 10 wherein the compound is Compound 1 or Compound 2; and X is a bond.

15. The pharmaceutical composition of claim 10 wherein the formulation is suitable for a route of administration selected from the group consisting of: oral, sublingual, oral inhalation, nasal inhalation, sublingual, rectal, vaginal, urethral, intravenous, intra-arterial, intradermal, intramuscular, subcutaneous, transdermal, mucosal and buccal.

16. The pharmaceutical composition of claim 10 wherein the release of the codrug is substantially controlled over an extended period of time selected from the group consisting of: about 4 hours, about 8 hours, about 12 hours, about 18 hours, about 24 hours, about 36 hours, about 48 hours, about 72 hours and about 96 hours.

17. The pharmaceutical composition of claim 16 wherein the release of the codrug is substantially controlled for about 6-12 hours.

18. The pharmaceutical composition wherein the release of the codrug is substantially controlled for about 12-24 hours.

19. A method of synthesis of a codrug comprising a linker, an opioid and a cannabinoid, said method comprising:
a) covalently bonding a first attachment point of the linker to the opioid;
b) covalently bonding a second attachment point of the linker to a cannabinoid; and
c) recovering the codrug.

20. The method of claim 19 further comprising:
a) reacting para-nitrophenyl chloroformate with an opiate (opioid) (R1) containing a hydroxy group in the presence of triethyl amine and dry chloroform;
b) cooling the solution;
c) recovering the resulting 6-O-para-nitrophenoxycarbonate ester of an opiate drug;
d) reacting the 6-O-para-nitrophenoxycarbonate ester of an opiate drug with a cannabinoid drug (R₂) with a hydroxyl group;
e) recovering the cannabinoid-opioid codrug.

21. The method of claim 14 comprising:
a) reacting para-nitrophenyl chloroformate with codeine to produce the para-nitrophenoxycarbonate ester of codeine;
b) reacting the para-nitrophenoxycarbonate ester of codeine with Δ-9 THC to produce 6-O, 1-O carbonate linked codrug of codeine and Δ-9 THC.

22. The method of claim 21 wherein step a) is reacted in the presence of dry chloroform and triethylamine.

23. The method of claim 21 wherein step a) occurs under cooled conditions and in a nitrogen atmosphere.

24. The method of claim 19 wherein step b) is reacted in the presence of dry THF and tri ethyl amine.

25. The method of claim 19 wherein step b) occurs under cooled conditions and in a nitrogen atmosphere.

26. A method of treatment comprising:
joining an opioid together with a cannabinoid using a linker to form a cleavable codrug; and
administering an analgesically effective amount of the codrug to a human patient.

21. The method of claim 26 wherein the codrug substantially remains intact until it reaches the site of action of at least the opioid or the cannabinoid.

27. The method of claim 26, wherein the codrug is more lipophilic than the opioid molecule.

28. The method of claim 26 wherein the codrug provides for a more desirable pharmacokinetic profile as compared to the opioid or cannabinoid when administered as distinct molecules.

29. The method of claim 26 wherein the amount of the opioid present in the codrug would be subtherapeutic if administered without the cannabinoid.

30. The method of claim 26 wherein the amount of the cannabinoid present in the codrug would be subtherapeutic if administered without the opioid.

31. The method of claim 26 wherein the amount of the cannabinoid and the amount of the opioid present in the codrug would each be subtherapeutic if not administered concomitantly.

32. The method of claim 26 wherein the synergistic analgesic effect of morphine and dronabinol is about 1.5-3 times greater than the extrapolated additive effect of administering morphine and dronabinol alone.

33. The method of claim 32 wherein the synergistic analgesic effect of morphine and dronabinol is about 2.5 times greater than the extrapolated additive effect of administering morphine and dronabinol alone.
